(19) United States
(12) Patent Application Publication
(10) Pub. No.: US 2009/0145201 A1
(43) Pub. Date: Jun. 11, 2009

(54) METHODS AND APPARATUSES FOR REDUCING EFFECTS OF MOLECULE ADSORPTION WITHIN MICROFLUIDIC CHANNELS

(75) Inventors: Hugh C. Crenshaw, Durham, NC (US); Kenneth I. Pettigrew, Sutherland, VA (US)

Correspondence Address: EKSIGENT TECHNOLOGIES, LLC;c/o SHELDON MAK ROSE & ANDERSON, 100 East Corson Street, Third Floor, PASADENA, CA, 91103-3842, US

(73) Assignee: EKSIGENT TECHNOLOGIES, LLC, Dublin, CA (US)

(21) Appl. No.: 11/719,530

(22) PCT Filed: Aug. 10, 2006
(86) PCT No.: PCT/US06/31165

§ 371 Date: May 16, 2007

Related U.S. Application Data
(60) Provisional application No. 60/707,366, filed on Aug. 11, 2005.

(51) Int. Cl. G01N 33/00

(52) U.S. Cl. 73/53.01; 422/68.1

(57) ABSTRACT

Microfluidic methods and apparatuses for reducing the effects of sample adsorption inside microfluidic chann are provided. According to one embodiment, a microfluidic chip (MFC) comprising an analysis channel (AC) having a cross-sectional area at least two times larger than a cross-sectional area of a microscale channel in fluid communication with the analysis channel is provided that reduces the effects of compound adsorption on data analysis. According to another embodiment, methods for reducing the effect of molecule adsorption to a channel wall (W) on analysis of a reaction in a microfluidic device and methods for making concentration dependent measurements in a microfluidic device are provided which utilize novel microfluidic chips disclose herein.

### CROSS-REFERENCE TO RELATED APPLICATIONS

This application claims the benefit of U.S. Patent Application Ser. No. 60/707,366, filed Aug. 11, 2005, the disclosure of which is incorporated herein by reference in its entirety. The disclosures of the following U.S. Provisional Applications, commonly owned and simultaneously filed Aug. 11, 2005, are all incorporated by reference in their entirety: U.S. Provisional Application entitled MICROFLUIDIC APPARATUS AND METHOD FOR SAMPLE PREPARATION AND ANALYSIS, U.S. Provisional Application No. 60/707,373 (Attorney Docket No. 447/99/2/1); U.S. Provisional Application entitled APPARATUS AND METHOD FOR HANDLING FLUIDS AT NANO-SCALE RATES, U.S. Provisional Application No. 60/707,421 (Attorney Docket No. 447/99/2/2); U.S. Provisional Application entitled MICROFLUIDIC BASED APPARATUS AND METHOD FOR THERMAL REGULATION AND NOISE REDUCTION, U.S. Provisional Application No. 60/707,330 (Attorney Docket No. 447/99/2/3); U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4); U.S. Provisional Application entitled METHODS AND APPARATUSES FOR GENERATING A SEAL BETWEEN A CONDUIT AND A RESERVOIR WELL, U.S. Provisional Application No. 60/707,286 (Attorney Docket No. 447/99/2/5); U.S. Provisional Application entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); U.S. Provisional Application entitled MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2); U.S. Provisional Application entitled MICROFLUIDIC SYSTEMS, DEVICES AND METHODS FOR REDUCING BACKGROUND AUTOFLUORESCENCE AND THE EFFECTS THEREOF, U.S. Provisional Application No. 60/707,386 (Attorney Docket No. 447/99/3/3); U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2); U.S. Provisional Application entitled METHODS FOR CHARACTERIZING BIOLOGICAL MOLECULE MODULATORS, U.S. Provisional Application No. 60/707,328 (Attorney Docket No. 447/99/5/1); U.S. Provisional Application entitled METHODS FOR MEASURING BIOCHEMICAL REACTIONS, U.S. Provisional Application No. 60/707,370 (Attorney Docket No. 447/99/5/2); U.S. Provisional Application entitled PLASTIC SURFACES AND APPARATUSES FOR REDUCED ADSORPTION OF SOLUTES AND METHODS OF PREPARING THE SAME, U.S. Provisional Application No. 60/707,288 (Attorney Docket No. 447/99/9); U.S. Provisional Application entitled BIOCHEMICAL ASSAY METHODS, U.S. Provisional Application No. 60/707,374 (Attorney Docket No. 447/99/10); U.S. Provisional Application entitled FLOW REACTOR METHOD AND APPARATUS, U.S. Provisional Application No. 60/707,233 (Attorney Docket No. 447/99/11); and U.S. Provisional Application entitled MICROFLUIDIC SYSTEM AND METHODS, U.S. Provisional Application No. 60/707,384 (Attorney Docket No. 447/99/12).

### TECHNICAL FIELD

The present disclosure generally relates to microfluidic processing of compounds and analysis of reaction products. More specifically, the present disclosure relates to microfluidic devices and methods for reduce the effects of compound adsorption inside microfluidic channels.

### BACKGROUND ART

Drug discovery requires many different measurements on molecules that have highly diverse chemical structures. Due to the large cost of drug discovery, there are many efforts to miniaturize the instruments and components used in these measurements, including the storage and handling of molecules.

A primary goal of miniaturization is to reduce the volume of reagent or solutions required for the process. Two consequences of miniaturization are that the ratio of surface area to volume increases, and the diffusion distance from the center of the volume to the surface of the volume also decreases.

As an example with a solution containing several solutes, if a first solute (S₁) in the solution adsorbs to the surface of the container, then the concentration of S₁ free in solution will decline with time. Subsequently, the ratio of S₁ in relation to all other components, such as a second solute (S₂) will change with time as well.

Many measurements made in drug discovery rely on knowledge of the concentration of a test molecule. Examples include:
The potency of a molecule, measured as the molecule's XC₅₀, defined as the concentration of the compound achieving 50% of maximal activity, inhibitory (IC₅₀) or excitatory (EC₅₀). Both have units of concentration. For example, a compound with an IC₅₀ of 200 nM against an enzyme will slow that enzyme to 50% of maximal enzymatic rate when the compound is at 200 nM; and
K Kinetic measures of an enzyme's action, such as the Michaelis constant, Kₘ, or the maximum rate of reaction, Vₘₐₓ.
   Adsorption of a test molecule to the wall of a microfluidic channel, therefore, can compromise any such measurements.

This phenomenon is most striking in microfluidic and other miniaturized systems in which the ratio of surface area to volume is many orders of magnitude larger than is found in more conventional approaches, such as for example, dispensing and mixing of solutions in microtiter plates. Thus, adsorption of molecules in microfluidic systems and other miniaturized devices can be a major. obstacle to miniaturization.

The problem of compound adsorption to surfaces potentially affects devices other than microfluidic channels. For example, compounds are typically stored, mixed, and studied in many different components such as pipette tips, microwells (such as in microtiter plates), tubes, vials, and other components. The adsorption of compounds to the surfaces of these components will affect the concentrations of those compounds in solution, especially if the concentration is low (to study potent compounds) and if the volume is small, which will occur in any miniaturization scheme.

One approach known in the art for decreasing the adsorption of solutes to the surfaces is to treat the surface—to alter its chemistry—so as to reduce adsorption. See Doherty et al., 2003 for a review of related applications in capillary electrophoresis. Alterations of surface chemistry have been used extensively to control the adsorption, or “sticking”, of proteins (see e.g. Rossier et al., 2000; Yang and Sundberg, 2001; and Becker and Locascio, 2002). One approach taken for proteins in microfluidic and other miniaturized systems has been to “PEGylate” the surface, that is to attach a layer of polyethylene glycol (PEG) to the surface (see e.g. Yang and Sundberg, 2001). PEGylation covers the surface with a hydrophilic material that ostensibly prevents adsorption of many biological proteins and cells, both prokaryotic and eukaryotic. Similar approaches have used detergents, especially non-ionic detergents, like the block copolymers, variably named “PLURONIC®” (BASF, Florham Park, N.J., U.S.A.) or “SYNPERONIC®” (ICI, New Castle, Del., U.S.A.), composed of blocks of polyethylene oxide-polypropylene oxide-polyethylene oxide (PEO-PPO-PEO) in which the hydrophilic PEO is similar to PEG (see Desai and Hubbell, 1991; Bridgett et al., 1992; Desai and Hubbell, 1992; Tan et al., 1993; Dewez et al, 1996; Dewez et al., 1997; Green et al., 1998; Detrait et al., 1999; Bromberg and Salvati, Jr., 1999; O'connor et al., 2000; Bevan and Prieve, 2000; Webb et al., 2001; Liu et al., 2002; Brandani and Stroeve, 2003; De Cupere et al., 2003; Musoke and Luckham, 2004).

These approaches have not proven completely effective. In fact, all attempts by applicants to PEGylate the surface of a microfluidic channel, to cover the surface with pluronics, or to otherwise make the surface more hydrophilic have actually made the surface even “stickier”, that is, adsorption is increased rather than decreased.

Another difficulty with engineering the surface chemistry is that the surface is typically designed to reduce specific chemical interactions with a single solute or a small number of solutes. However, a primary practice in drug discovery is to use a large and chemically diverse library of compounds. This prevents such tailored engineering.

It is desirable, therefore, in miniaturization to have device architectures and surface chemistries in which adsorption of solutes, especially drug-like organic molecules, is minimized.

### SUMMARY

According to a first embodiment of the presently disclosed subject matter, a microfluidic analysis channel is provided. In one embodiment, the analysis channel comprises an inlet having a first cross-sectional area for passage of fluid therethrough; and an analysis region in fluid communication with the inlet and having a second cross-sectional area for passage of fluid from the inlet to the analysis region, the second cross-sectional area being greater than the first cross-sectional area, whereby adsorption of a compound in fluid in the analysis region is decreased and a reduction of concentration of the compound at a center axis region in the analysis region is minimized.

According to a second embodiment of the presently disclosed subject matter, a microfluidic device is provided. In some embodiments, the microfluidic device comprises at least one microscale channel for passage of fluid therethrough having a first cross-sectional area; and an analysis channel in fluid communication with the microscale channel and having a second cross-sectional area, the second cross-sectional area being greater than the first cross-sectional area, whereby adsorption of a compound in fluid in the analysis channel is decreased and a reduction of concentration of the compound at a center axis region in the analysis channel is minimized. In some embodiments, the microfluidic device comprises a controlled dispersion element in fluid communication with and located upstream of the analysis channel. In some embodiments, the controlled dispersion element is an expansion channel. In some embodiments, the microfluidic device is comprised of a polymer, quartz, or silicon.

In some embodiments, the analysis channel can further comprise an expansion region beginning at the inlet and having an upstream cross-sectional area approximately equivalent to the inlet first cross-sectional area and a downstream cross-sectional area approximately equivalent to the second cross-sectional area. In some embodiments, the downstream cross-sectional area is at least two times greater than the upstream cross-sectional area and in some embodiments, the downstream cross-sectional area is between about two times and about five hundred times larger than the upstream cross-sectional area.

In some embodiments, the analysis region has an aspect ratio of height to width equal to 1. In some embodiments, the analysis region further comprises a detection area located along at least a portion of a center axis region of the analysis region.

According to a third embodiment of the presently disclosed subject matter, a method for decreasing adsorption of a compound in a fluid and minimizing reduction of concentration of the compound in a microfluidic device is provided. In some embodiments, the method comprises providing a microfluidic device comprising an analysis channel, which comprises: an inlet having a first cross-sectional area for passage of fluid therethrough; and an analysis region in fluid communication with the inlet and having a second cross-sectional area for passage of fluid from the inlet to the analysis region, the second cross-sectional area being greater than the first cross-sectional area; and passing a fluid comprising a compound through the inlet and into the analysis region, whereby adsorption of the compound in the fluid in the analysis region is decreased and a reduction of concentration of the compound at a center axis region in the analysis region is minimized.

In some embodiments, the method further comprises analyzing the compound or a product resulting from interaction of the compound with at least one other compound in the detection area. In some embodiments, analyzing the compound or the product utilizes confocal optics focused at the detection area. Further, in some embodiments, analyzing the compound or the product comprises determining steady-state kinetic constants; Michaelis constants (Kₘ); kinetic isotope effects on enzyme catalyzed reactions; dose-responses of inhibitors or activators on enzyme or receptor activity (IC₅₀ and EC₅₀ value); mechanisms of inhibition of an enzyme catalyzed reaction and associated inhibition constants (slope inhibition constant (Kᵢₛ) and intercept inhibition constant (Kᵢᵢ)); interaction factors between multiple inhibitors (α); kinetic mechanisms of multi-substrate enzyme reactions; capacity of receptor binding (Bₘₐₓ); pH effects on enzyme catalysis; pH effects on enzyme binding; binding constants (K_{d}); binding stoichiometry; or combinations thereof.

In some embodiments, the compound or the product is fluorescently labeled. Further, in some embodiments where the compound is fluorescently labeled, analyzing the compound or the product comprises measuring fluorescence intensity, polarization fluorescence, fluorescence resonance energy transfer (FRET), fluorescence lifetime, or combinations thereof.

According to a fourth embodiment of the presently disclosed subject matter, a method for making concentration dependent measurements in a microfluidic device is provided. In some embodiments, the method comprises flowing a fluid stream comprising at least one compound through at least one microscale channel of a microfluidic device; continuously varying the concentration of the compound within the fluid stream; flowing the fluid stream through an analysis channel in fluid communication with the microscale channel, the analysis channel comprising: an inlet having a first cross-sectional area for passage of the fluid stream therethrough; an analysis region in fluid communication with the inlet and having a second cross-sectional area for passage of the fluid stream from the inlet to the analysis region, the second cross-sectional area being greater than the first cross-sectional area, whereby adsorption of the compound in the fluid stream in the analysis region is decreased and a reduction of concentration of the compound at a center axis region in the analysis region is minimized; and a detection area located within the analysis region; and measuring the fluid stream at the detection area along at least a portion of the continuously varying concentration gradient of the compound.

In some embodiments, flowing the fluid stream comprising at least one compound through at least one microscale channel of the microfluidic device comprises a first compound flowing within a first fluid stream through a first microfluidic channel and a second compound flowing within a second fluid stream through a second microfluidic channel. In some embodiments, the first and second microfluidic channels merge at a merge region, thereby flowing the first fluid stream into contact with the second fluid stream to form a merged fluid stream.

In some embodiments of the method, continuously varying the concentration of the compound within the fluid stream comprises creating a continuous concentration gradient for the first and second compounds through controlled variation of volumetric flow rates of the first and second fluid streams. In some embodiments, the first and second fluid streams are driven by a first and second pump, respectively. Further, in some embodiments, varying volumetric flow rate of the first and second fluid streams comprises controlling speeds of the first pump and the second pump, respectively. Still further, in some embodiments, the first and second pumps can be synchronized to maintain an overall constant volumetric flow rate while varying individual volumetric flow rates of the first and second fluid streams.

In some embodiments, the method comprises a third compound flowing within a third fluid stream through a third microfluidic channel, wherein the third fluid stream merges with the merged fluid stream at a second merge region. In some embodiments, a time in transit from the second merge region to the analysis channel is sufficient to permit diffusional mixing of the compounds. Further, in some embodiments, the volumetric flow rate of the third pump is constant. Still further, in some embodiments, the combined volumetric flow rate of the three pumps is constant.

In some embodiments, the first, second, and third pumps are displacement pumps. Further, in some embodiments, controlling speeds of the first and second pumps comprises reducing sharp transitions within the continuous concentration gradient. Still further, in some embodiments, controlling speeds of the first and second pumps comprises creating a continuous concentration gradient having multiple slopes. Even further, in some embodiments, controlling speeds of the first and second pumps comprises creating a logarithmic continuous concentration gradient.

In some embodiments, measuring the fluid stream comprises determining steady-state kinetic constants; Michaelis constants (Kₘ), kinetic isotope effects on enzyme catalyzed reactions; dose-responses of inhibitors or activators on enzyme or receptor activity (IC₅₀ and EC₅₀ value); mechanisms of inhibition of an enzyme catalyzed reaction and associated inhibition constants (slope inhibition constant (Kᵢₛ) and intercept inhibition constant (Kᵢᵢ)); interaction factors between multiple inhibitors (α); kinetic mechanisms of multi-substrate enzyme reactions; capacity of receptor binding (Bₘₐₓ); pH effects on enzyme catalysis; pH effects on enzyme binding; binding constants (K_{d}); binding stoichiometry; or combinations thereof.

In some embodiments, the compound is fluorescently labeled. Further, in some embodiments where the compound is fluorescently labeled, measuring the fluid stream comprises measuring fluorescence intensity, polarization fluorescence, fluorescence resonance energy transfer (FRET), fluorescence lifetime, or combinations thereof.

Therefore, it is an object to provide devices and methods for reducing effects of molecule adsorption within microfluidic channels.

An object of the presently disclosed subject matter having been stated hereinabove, and which are achieved in whole or in part by the present disclosure, other objects will become evident as the description proceeds when taken in connection with the accompanying drawings as best described hereinbelow.

### BRIEF DESCRIPTION OF THE DRAWINGS

FIG. 1 is a schematic view of a sample processing apparatus including a pump assembly and a microfluidic chip provided in accordance with embodiments disclosed herein;

FIG. 2 is a simplified diagram of a linear displacement pump provided in embodiments of sample processing apparatuses disclosed herein;

FIG. 3A is a plot of step gradients generated by two pumps, each containing a different fluorophore, and controlled to create steps of 0.1 nl/min ranging from 0.0 to 1.0 nl/min;

FIG. 3B is a plot of pump-driven flow velocity profiles superimposed over a plot of a measured concentration value resulting from the combination of reagent input streams in accordance with the flow velocity profiles according to embodiments disclosed herein;

FIG. 4 is a schematic view of a sample processing apparatus with sample measurement components integrated therein according to embodiments disclosed herein;

FIG. 5 is a schematic view of a fluorescence measurement apparatus provided in accordance with embodiments disclosed herein;

FIG. 6 is a graph showing an exemplary experiment measuring IC₅₀ using an embodiment of a sample processing apparatus as disclosed herein;

FIG. 7 is a characterization of adsorption of compounds to a wall of a microfluidic channel;

FIG. 8 is a graph showing data from FIG. 6 transformed to concentration versus enzyme activity;

FIG. 9 is a schematic top view of an embodiment of an analysis channel disclosed herein and upstream fluidly communicating microscale channels;

FIG. 10A is a schematic cross-sectional side view of an embodiment of analysis channel disclosed herein and upstream fluidly communicating microscale channel;

FIG. 10B shows schematic cross-sectional cuts at A-A and B-B of the analysis channel of FIG. 10A;

FIG. 11 is a schematic view of a sample processing apparatus including a pump assembly, a microfluidic chip, and a novel analysis channel provided in accordance with embodiments disclosed herein;

FIG. 12 is a graph showing an exemplary experiment measuring IC₅₀ using an embodiment of a sample processing apparatus as disclosed herein comprising a novel analysis channel;

FIG. 13 is a graph showing data from FIG. 12 transformed to concentration versus enzyme activity;

FIG. 14 is a graph showing an exemplary experiment measuring IC₅₀ using an embodiment of a sample processing apparatus as disclosed herein comprising a novel analysis channel and further showing a magnified portion of the two traces showing the very low noise of the data;

FIG. 15 is a graph overlapping data from FIGS. 6 and 12 for comparison;

FIG. 16 is a graph showing the extent of dispersion of compounds in a mixture over time;

FIG. 17 is a schematic top view of an embodiment of an analysis channel disclosed herein and upstream fluidly communicating microscale channels and expansion channel;

FIG. 18 is a graph of an exponential continuously variable concentration gradient;

FIG. 19 is a graph of a continuously variable concentration gradient exhibiting three distinct slopes;

FIG. 20 is a graph plotting the ratio D/D′ for channels of different diameters at a volumetric flow rate of 30 nl/min; and

FIG. 21 is a graph plotting psi vs. k for predefined flow.

### DETAILED DESCRIPTION

Microfluidic chips, systems, and related methods are described herein which incorporate improvements for reducing or eliminating effects of molecule adsorption within microfluidic channels. These microfluidic chips, systems, and methods are described with regard to the accompanying drawings. It should be appreciated that the drawings do not constitute limitations on the scope of the disclosed microfluidic chips, systems, and methods.

As used herein, the term “microfluidic chip,” “microfluidic system,” or “microfluidic device” generally refers to a chip, system, or device which can incorporate a plurality of interconnected channels or chambers, through which materials, and particularly fluid borne materials can be transported to effect one or more preparative or analytical manipulations on those materials. A microfluidic chip is typically a device comprising structural or functional features dimensioned on the order of mm-scale or less, and which is capable of manipulating a fluid at a flow rate on the order of μl/min or less. Typically, such channels or chambers include at least one cross-sectional dimension that is in a range of from about 1 μm to about 500 μm. The use of dimensions on this order allows the incorporation of a greater number of channels or chambers in a smaller area, and utilizes smaller volumes of reagents, samples, and fluids for performing the preparative or analytical manipulation of the sample that is desired.

Microfluidic systems are capable of broad application and can generally be used in the performance of biological and biochemical analysis and detection methods. The systems described herein can be employed in research, diagnosis, environmental assessment and the like. In particular, these systems, with their micron scales, nanoliter volumetric fluid control systems, and integratability, can generally be designed to perform a variety of fluidic operations where these traits are desirable or even required. In addition, these systems can be used in performing a large number of specific assays that are routinely performed at a much larger scale and at a much greater cost.

A microfluidic device or chip can exist alone or may be a part of a microfluidic system which, for example and without limitation, can include: pumps for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, current and the like.

As used herein, the term “channel” or “microfluidic channel” can mean a cavity formed in a material by any suitable material removing technique, or can mean a cavity in combination with any suitable fluid-conducting structure mounted in the cavity such as a tube, capillary, or the like.

As used herein, the terms “compound” and “reagent” are used interchangeably and generally mean any flowable composition or chemistry. The result of two compounds or reagents merging or combining together is not limited to any particular response, whether a biological response or biochemical reaction, a dilution, or otherwise.

In referring to the use of a microfluidic chip for handling the containment or movement of fluid, the terms “in”, “on”, “into”, “onto”, “through”, and “across” the chip generally have equivalent meanings.

As used herein, the term “communicate” (e.g., a first component “communicates with” or “is in communication with” a second component) and grammatical variations thereof are used herein to indicate a structural, functional, mechanical, electrical, optical, or fluidic relationship, or any combination thereof, between two or more components or elements. As such, the fact that one component is said to communicate with a second component is not intended to exclude the possibility that additional components may be present between, and/or operatively associated or engaged with, the first and second components.

As used herein, the terms “measuring”, “sensing”, and “detecting” and grammatical variations thereof have interchangeable meanings; for the purpose of the present disclosure, no particular distinction among these terms is intended.

As used herein, the term “fluid” generally means any flowable medium such as liquid, gas, vapor, supercritical fluid, combinations thereof, or the ordinary meaning as understood by those of skill in the art.

As used herein, the term “vapor” generally means any fluid that can move and expand without restriction except for at a physical boundary such as a surface or wall, and thus can include a gas phase, a gas phase in combination with a liquid phase such as a droplet (e.g., steam), supercritical fluid, the like, or the ordinary meaning as understood by those of skill in the art.

Embodiments disclosed herein comprise hardware and/or software components for controlling liquid flows in microfluidic devices and measuring the progress of miniaturized biochemical reactions occurring in such microfluidic devices. As the description proceeds, it will become evident that the various embodiments disclosed herein can be combined according to various configurations to create a technologic system or platform for implementing micro-scale or sub-micro-scale analytical functions. One or more of these embodiments can contribute to or attain one or more advantages over prior art technology, including: (1) 1000-fold reduction in the amount of reagent needed for a given assay or experiment; (2) elimination of the need for disposable assay plates; (3) fast, serial processing of independent reactions; (4) data readout in real-time; (5) improved data quality; (6) more fully integrated software and hardware, permitting more extensive automation of instrument function, 24/7 operation, automatic quality control and repeat of failed experiments or bad gradients, automatic configuration of new experimental conditions, and automatic testing of multiple hypotheses; (7) fewer moving parts and consequently greater robustness and reliability; and (8) simpler human-instrument interface. As the description proceeds, other advantages may be recognized by persons skilled in the art.

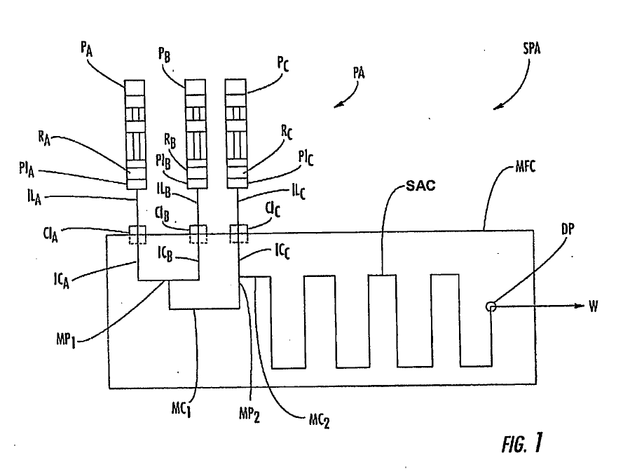

Referring now to FIG. 1, a sample processing apparatus, generally designated SPA, is illustrated according to certain embodiments. Generally, sample processing apparatus SPA can be utilized for precisely generating and mixing continuous concentration gradients of reagents in the nl/min to μl/min range, particularly for initiating a biological response or biochemical reaction from which results can be read after a set period of time. Sample processing apparatus SPA can generally comprise a reagent introduction device that can be provided in the form of a pump assembly, generally designated PA, and a microfluidic device or chip MFC. Pump assembly PA can comprise one or more linear displacement pumps such as syringe pumps or the like. For mixing two or more reagents, pump assembly PA can comprise at least two or more pumps. In the illustrated embodiment in which three reagents can be processed (e.g., reagent R_{A}, R_{B}, and R_{C}), sample processing apparatus SPA can include a first pump P_{A}, a second pump P_{B}, and a third pump P_{C}. Sample processing apparatus SPA can be configured such that pumps P_{A}, P_{B} and P_{C} are disposed off-chip but inject their respective reagents R_{A}, R_{B} and R_{C} directly into microfluidic chip MFC via separate input lines IL_{A}, IL_{B} and IL_{C} such as fused silica capillaries, polyetheretherketone (such as PEEK® available from Upchurch Scientific of Oak Harbor, Wash., U.S.A.) tubing, or the like. In some embodiments, the outside diameter of input lines IL_{A}, IL_{B} and IL_{C} can range from approximately 50-650 μm. In some embodiments, each pump P_{A}, P_{B} and P_{C} can interface with its corresponding input line IL_{A}, IL_{B} and IL_{C} through a pump interconnect PI_{A}, PI_{B} and PI_{C} designed for minimizing dead volume and bubble formation, and with replaceable parts that are prone to degradation or wear. Pump interconnects PI_{A}, PI_{B} and PI_{C} according to some embodiments are described in more detail in co-pending, commonly owned U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2); the content of which is incorporated herein in its entirety.

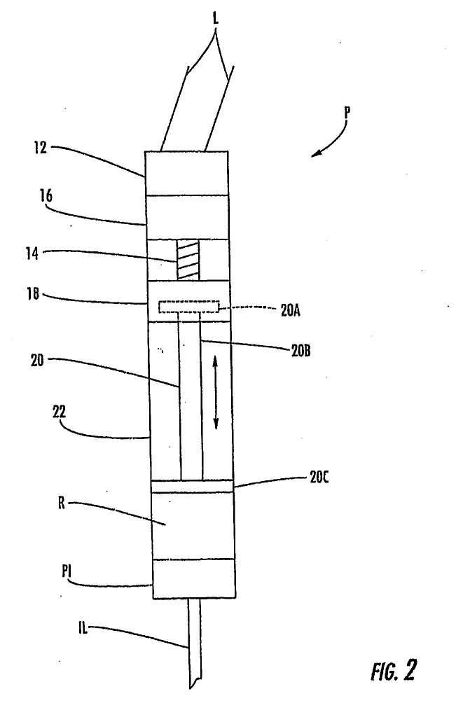

Referring to FIG. 2, an example of a suitable linear displacement pump, generally designated P, is diagrammatically illustrated. Pump P can include a servo motor 12 that can be energized and controlled through its connection with any suitable electrical circuitry, which could comprise computer hardware and/or software, via electrical leads L. Alternatively, pump P can include any suitable motor for driving the components of a linear displacement pump. For example, pump P can be a stepper motor. As shown, servo motor 12 drives a rotatable lead screw 14 through a gear reduction device 16. Lead screw 14 engages a linearly translatable pump stage 18. A piston or plunger 20 is coupled to pump stage 18 for linear translation within a pump barrel 22 that stores and contains a reagent R to be introduced into microfluidic chip MFC (FIG. 1). Typically, plunger 20 can comprise a head portion 20A, an elongate portion or stem 20B, and a distal end or movable boundary 20C. In operation, reagent R is pushed by movable boundary 20C through pump interconnect PI and into input line IL. The structure of each pump P according to advantageous embodiments is further described in co-pending, commonly owned U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4), the content of which is incorporated herein in its entirety.

In one exemplary yet non-limiting embodiment and as shown in FIG. 2, pump barrel 22 can be a gas-tight micro-syringe type, having a volume ranging from approximately 10-250 μl. The thread pitch of lead screw 14 can be approximately 80 threads per inch. Gear reduction device 16 produces a gear reduction of 1024:1 or thereabouts. Servo motor 12 and gear reduction device 16 can have an outside diameter of 10 mm or thereabouts. Servo motor 12 uses a 10-position magnetic encoder with quadrature encoding that provides forty encoder counts per revolution, and the resolution is such that each encoder count is equivalent to 0.0077 μm of linear displacement. The foregoing specifications for the components of pump P can be changed without departing from the scope of the embodiment.

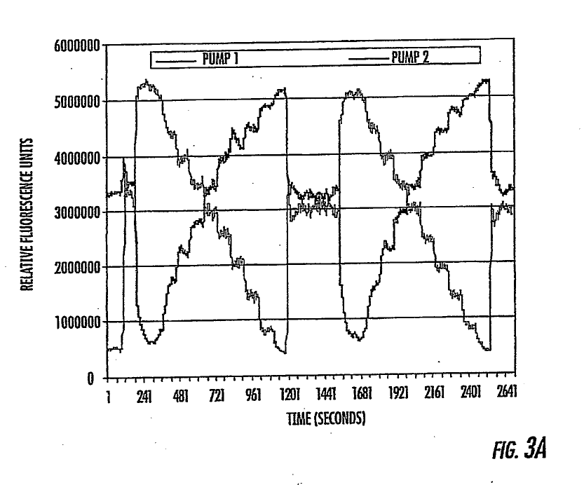

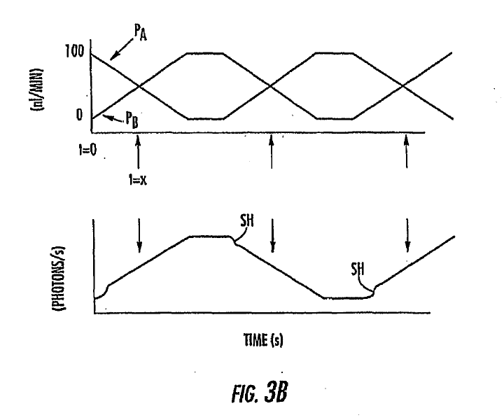

In some embodiments for which a plurality of pumps are provided (e.g., pumps P_{A}-P_{C} in FIG. 1), the respective operations of pumps P_{A}-P_{C} and thus the volumetric flow rates produced thereby can be individually controllable according to individual, pre-programmable fluid velocity profiles. The use of pumps P_{A}-P_{C} driven by servo motors 12 can be advantageous in that smooth, truly continuous (i.e., non-pulsatile) flows can be processed in a stable manner. In some embodiments, pumps P_{A}-P_{C} are capable of producing flow rates permitting flow grading between about 0 and 500 nl/min, with a precision of 0.1 nl/min in a stable, controllable manner. Optionally, pumps P_{A}-P_{C} can produce flow rates permitting flow grading from 0 to as little as 5 nl/min. FIG. 3A is a plot of step gradients generated by two pumps, each containing a different fluorophore, and controlled to create steps of 0.1 nl/min ranging from 0.0 to 1.0 nl/min. The flow in the two pumps was merged in a microfluidic chip and the resulting fluorescence signals were measured to determine the ratio of the mix. The combined flow rate of the two pumps was 1 nl/min, with steps of 0.1 nl/min being made to demonstrate the precision of the flow rate. Continuously varying flows also are possible, as described hereinbelow. Moreover, the operation of each servo motor 12 (e.g., the angular velocity of its rotor) can be continuously varied in direct proportion to the magnitude of the electrical control signal applied thereto. In this manner, the ratio of two or more converging streams of reagents (e.g., reagents R_{A}-R_{C} in FIG. 1) can be continuously varied over time to produce continuous concentration gradients in microfluidic chip MFC. Thus, the number of discrete measurements that can be taken from the resulting concentration gradient is limited only by the sampling rate of the measurement system employed and the noise in the concentration gradient. Moreover, excellent data can be acquired using a minimal amount of reagent. For instance, in the practice of the present embodiment, high-quality data has been obtained from concentration gradients that consumed only 100 nl of reagent (total volume) from three simultaneous flows of reagents R_{A}-R_{C}.

The ability to produce very low flow-rate, stable displacement flows to generate concentration gradients, believed to be 3-4 orders of magnitude slower than that heretofore attainable, provides a number of advantages. Chips can be fabricated from any material, and surface chemistry does not need to be carefully controlled, as with electro-osmotic pumping. Any fluid can be pumped, including fluids that would be problematic for electro-osmotic flows (full range of pH, full range of ionic strength, high protein concentrations) and for pressure driven flows (variable viscosities, non-Newtonian fluids), greatly simplifying the development of new assays. Variations in channel diameters, either from manufacture variability or from clogging, do not affect flow rates, unlike electro-osmotic or pressure flows. Computer control and implementation of control (sensors and actuators) are simpler than for pressure flows, which require sensors and actuators at both ends of the channel. Displacement-driven flows provide the most-straighfforward means for implementing variable flows to generate concentration gradients.

The ability to pump at ultra-low flow rates (nl/min) provides a number of advantages in the operation of certain embodiments of microfluidic chip MFC and related methods disclosed herein. These low flow rates enable the use of microfluidic channels with very small cross-sections. Higher, more conventional flow rates require the use of longer channels in order to have equivalent residence times (required to allow many biochemical reactions or biological responses to proceed) or channels with larger cross-sectional areas (which can greatly slow mixing by diffusion and increase dispersion of concentration gradients). In addition, reagent use is decreased because, all other parameters being equal, decreasing the flow rate by half halves the reagent use. Smaller channel dimensions (e.g., 5-30 μm) in the directions required for diffusional mixing of reagents permits even large molecules to rapidly mix in the microfluidic channels.

Referring again to FIG. 1, microfluidic chip MFC comprises a body of material in which channels are formed for conducting, merging, and mixing reagents R_{A}-R_{C} for reaction, dilution or other purposes. Microfluidic chip MFC can be structured and fabricated according to any suitable techniques, and using any suitable materials, now known or later developed. In advantageous embodiments, the channels of microfluidic chip MFC can be formed within its body to prevent evaporation, contamination, or other undesired interaction with or influence from the ambient environment.

Suitable examples of such a microfluidic chip MFC are disclosed in co-pending, commonly owned U.S. Provisional Applications entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2); MICROFLUIDIC SYSTEMS, DEVICES AND METHODS FOR REDUCING BACKGROUND AUTOFLUORESCENCE AND THE EFFECTS THEREOF, U.S. Provisional Application No. 60/707,386 (Attorney Docket No. 447/99/3/3); and MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2), the contents of which are incorporated herein in their entireties. As described therein, to provide internal channels, microfluidic chip MFC can comprise two body portions such as plates or layers, with one body portion serving as a substrate or base on which features such as channels are formed and the other body portion serving as a cover. The two body portions can be bonded together by any means appropriate for the materials chosen for the body portions. Non-limiting examples of bonding techniques can include thermal bonding, anodic bonding, glass frit bonding, adhesive bonding, and the like. Non-limiting examples of materials used for the body portions can include various structurally stable polymers such as polystyrene and polycarbonate, metal oxides such as sapphire (Al₂O₃), silicon, and oxides, nitrides or oxynitrides of silicon (e.g., SiₓN_{y}, glasses such as SiO₂, or the like). In advantageous embodiments, the materials can be chemically inert and biocompatible relative to the reagents to be processed, or can include surfaces, films, and coatings or are otherwise treated so as to be rendered inert and/or biocompatible. The body portions can be constructed from the same or different materials. To enable optics-based data encoding of analytes processed by microfluidic chip MFC, one or both body portions can be optically transmissive or include windows at desired locations. The channels can be formed by any suitable micro-fabricating techniques appropriate for the materials used, such as the various etching, masking, photolithography, ablation, and micro-drilling techniques available. The channels can be formed, for example, according to the methods disclosed in a co-pending, commonly owned U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2), the content of which is incorporated herein in its entirety. In some embodiments, the size of the channels can range from approximately 5 to 500 μm in cross-sectional area.

As shown in FIG. 1, as one exemplary fluidic architecture, the channels of microfluidic chip MFC can include a first input or pre-mixing channel IC_{A}, a second input or pre-mixing channel IC_{B}, and a third input or pre-mixing channel IC_{C}. Input channels IC_{A}, IC_{B} and IC_{C} are shown fluidly communicating with corresponding pumps P_{A}, P_{B}, and P_{C} via input lines IL_{A}, IL_{B}, and IL_{C}. In some embodiments, input channels IC_{A}, IC_{B} and IC_{C} can interface with input lines IL_{A}, IL_{B}, and IL_{C} through respective chip interconnects CI_{A}, CI_{B} and CI_{C}. Chip interconnects CI_{A}, CI_{B} and CI_{C} can be provided in accordance with embodiments disclosed in a co-pending, commonly owned U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2), the content of which is incorporated herein in its entirety. In addition to introducing separate reagent streams into microfluidic chip MFC, first and second input channels IC_{A} and IC_{B} can serve as temperature-equilibrating channels in which their respective reagents R_{A} and R_{B} to be mixed are equilibrated to a given surrounding temperature.

First input channel IC_{A} through which a first fluid stream can flow and second input channel IC_{B} through which a second fluid stream can flow terminate or meet at a first T-junction region or merging point MP₁. From first merging point MP₁, a first mixing channel MC₁ traverses through microfluidic chip MFC over a distance sufficient to enable passive mixing within a merged fluid stream of reagents R_{A} and R_{B} introduced by first input channel IC_{A} and second input channel IC_{B}. In some embodiments, the mechanism for passive mixing is thermal or molecular diffusion that depends on time and diffusion distance. Accordingly, microfabricated active mixers, which can be a source of noise, complexity, unreliability and cost are not required but could be provided. In the present exemplary embodiment, third input channel IC_{C} and first mixing channel MC₁ terminate or meet at a second T-junction region or merging point MP₂, from which a second mixing channel MC₂ traverses through microfluidic chip MFC over a distance sufficient for mixing.

Second mixing channel MC₂ communicates with a process/reaction channel or aging loop AL. Aging loop AL has a length sufficient for prosecuting a reaction or other interaction between reagents after the reagents have been introduced in two or more of first input channel IC_{A}, second input channel IC_{B} and/or third input channel IC_{C}, merged at first mixing point MP₁ and/or second mixing point MP₂, and thereafter mixed in first mixing channel MC₁ and/or second mixing channel MC₂. For a given area of microfluidic chip MFC, the length of aging loop AL can be increased by providing a folded or serpentine configuration as illustrated in FIG. 1. For many processes contemplated herein, the length of aging loop AL and the linear velocity of the fluid flowing therethrough determines the time over which a reaction can proceed. A longer aging loop AL or a slower linear velocity permits longer reactions. The length of aging loop AL can be tailored to a specific reaction or set or reactions, such that the reaction or reactions have time to proceed to completion over the length of aging loop AL. Conversely, a long aging loop AL can be used in conjunction with measuring shorter reaction times by taking measurements closer to second mixing channel MC₂.

As further illustrated in FIG. 1, a detection area or point DP is defined in microfluidic chip MFC at an arbitrary point along the flow path of the reagent mixture, e.g., at a desired point along aging loop AL. More than one detection area or point DP can be defined so as to enable multi-point measurements and thus permit, for example, the measurement of a reaction product at multiple points along aging loop AL and hence analysis of time-dependent phenomena or automatic localization of the optimum measurement point (e.g., finding a point yielding a sufficient yet not saturating analytical signal). In some methods as further described hereinbelow, however, only a single detection area or point DP may be needed. Detection point DP represents a site of microfluidic chip MFC at which any suitable measurement (e.g., concentration) of the reagent mixture can be taken by any suitable encoding and data acquisition technique. As one example, an optical signal can be propagated though microfluidic chip MFC at detection point DP, such as through its thickness (e.g., into or out from the sheet of FIG. 1) or across its plane (e.g., toward a side of the sheet of FIG. 1), to derive an analytical signal for subsequent off-chip processing. Hence, microfluidic chip MFC at detection point DP can serve as a virtual, micro-scale flow cell as part of a sample analysis instrument.

After an experiment has been run and data have been acquired, the reaction products flow from aging loop AL to any suitable off-chip waste site or receptacle W. Additional architectural details and features of microfluidic chip MFC are disclosed in co-pending, commonly owned U.S. Provisional Applications entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2); MICROFLUIDIC SYSTEMS, DEVICES AND METHODS FOR REDUCING BACKGROUND AUTOFLUORESCENCE AND THE EFFECTS THEREOF, U.S. Provisional Application No. 60/707,386 (Attorney Docket No. 447/99/3/3); and MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2), the contents of which are incorporated in their entireties.

An example of a method for generating and mixing concentration gradients using sample processing apparatus SPA illustrated in FIG. 1 will now be described. The respective pump barrels 22 (FIG. 2) of two or more of pumps P_{A}-P_{C} are filled with different reagents R_{A}-R_{C} and installed in pump assembly PA (FIG. 1). It will be understood, however, that one or more of pumps P_{A}-P_{C} could be placed in communication with an automated or non-automated liquid handling system to selectively supply reagents R_{A}-R_{C} as well as buffers, solvents, and the like. Examples of automated liquid handling systems are described in co-pending, commonly owned U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4) and U.S. Provisional Application entitled APPARATUS AND METHOD FOR HANDLING FLUIDS AT NANO-SCALE RATES, U.S. Provisional Application No. 60/707,421 (Attorney Docket No. 447/99/2/2), the contents of which are incorporated herein in their entirety. Microfluidic chip MFC, typically with input lines IL_{A}, IL_{B} and IL_{C} attached, can be mounted to any suitable holder such as a microscope stage as described hereinbelow in conjunction with one particular embodiment. The proximal (upstream) ends of input lines IL_{A}, IL_{B} and IL_{C} can be attached to the corresponding distal (downstream) ends of pump barrels 22 (FIG. 2), such as by using pump interconnects PI_{A}-PI_{C} according to certain embodiments disclosed herein. Any suitable method can then be performed to purge the channels of microfluidic chip MFC to remove any contaminants, as well as bubbles or any other compressible fluids affecting flow rates and subsequent concentration gradients. For instance, prior to loading reagents R_{A}-R_{C} into pump assembly PA, pump assembly PA can be used to run a solvent through microfluidic chip MFC. Any configuration and calibration of the equipment used for detection/measurement can also be performed at this point, including the selection and/or alignment of optical equipment such as, for example confocal optics, described hereinbelow.

Once sample processing apparatus SPA has been prepared, concentration gradients can be run through microfluidic chip MFC. Two or more of pumps P_{A}, P_{B} and/or P_{C} can be activated to establish separate flows of different reagents R_{A}, R_{B} and/or R_{C} into microfluidic chip MFC for combination, mixing, reaction, and measurement. A variety of combining strategies can be employed, depending on the number of inputs into microfluidic chip MFC and the corresponding number of pumps P_{A}-P_{C}, on their sequence of mixing determined by the geometry of fluidic channels in microfluidic chip MFC, and on the sequence of control commands sent to the pumps P_{A}-P_{C}. Using a microfluidic chip MFC with three inputs as illustrated in FIG. 1, for example, three reagents (reagents R_{A}, R_{B} and R_{C}) can be input into microfluidic chip MFC, and concentration gradients of reagents R_{A} versus R_{B} can then be run against a constant concentration of reagent R_{C}. For another example, by using a four-input microfluidic chip MFC, concentration gradients of reagents R_{A} and R_{B} can be run with fixed concentrations of reagent R_{C} and an additional reagent R_{D}. Due to the small size of the channels of microfluidic chip MFC, reagents R_{A}, R_{B} and/or R_{C} mix quickly (e.g., less than one second) in mixing channels MC₁ and/or MC₂ due to passive diffusion.

In accordance with one embodiment of the method, the total or combined volumetric flow rate established by the active pumps P_{A}, P_{B} and/or P_{C} can be maintained at a constant value during the run, in which case the transit time from mixing to measurement is constant and, consequently, the duration of reaction is held constant. In addition, the ratio of the individual flow rates established by respective pumps P_{A}, P_{B} and/or P_{C} can be varied over time by individually controlling their respective servo motors 12, thereby causing the resulting concentration gradient of the mixture in aging loop AL to vary with time (i.e. concentration varies with distance along aging loop AL). The concentration gradient of interest is that of the analyte relative to the other components of the mixture. The analyte can be any molecule of interest, and can be any form of reagent or component. Non-limiting examples include inhibitors, substrates, enzymes, fluorophores or other tags, and the like. As the reaction product passes through detection point DP with a varying concentration gradient, the detection equipment samples the reaction product flowing through according to any predetermined interval (e.g., 100 times per second). The measurements taken of the mixture passing through detection point DP can be temporally correlated with the flow ratio produced by pumps P_{A}, P_{B} and/or P_{C}, and a response can be plotted as a function of time or concentration.

Referring to FIG. 3B, an exemplary plot of varying flow velocity profiles programmed for two pumps (e.g., pumps P_{A} and P_{B}) is given as a function of time, along with the resulting reagent concentration over time. As can be appreciated by persons skilled in the art, the flow velocity profiles can be derived from information generated by encoders typically provided with pumps P_{A}, P_{B} and P_{C} that, for example, transduce the angular velocities of their respective servo motors 12 by magnetic coupling or by counting a reflective indicator such as a notch or hash mark. Similarly, a linear encoder can directly measure the movement of plunger 20 or parts that translate with plunger 20. It can be seen that the total volumetric flow rate can be kept constant even while varying concentration gradients over time, by decreasing the flow rate of pump P_{A} while increasing the flow rate of pump P_{B}. For instance, at time t=0, the flow rate associated with pump P_{A} has the relative value of 100% of the total volumetric flow rate, and the flow rate associated with pump P_{B} has the relative value of 0%. As the flow rate of pump P_{A} is ramped down and the flow rate of pump P_{B} is ramped up, their respective profile lines cross at time t=x, where each flow rate is 50%. As shown in FIG. 3B, each flow rate can be oscillated between 0% and 100%. The resulting plot of concentration can be obtained, for example, through the use of a photodetector that counts photons per second, although other suitable detectors could be utilized as described hereinbelow. Similarly, non-linear concentration gradients and more complex concentration gradients of reagents R_{A}, R_{B} and R_{C} can be generated through appropriate command of the pumps P_{A}, P_{B} and P_{C}. The trace of fluorescence in FIG. 3B includes apparent steps of “shoulders” SH at the beginning of each increasing gradient and each decreasing gradient. These can arise from such phenomena as stiction in the pump or associated parts, inertia of the motor, poor encoder resolution at rotational velocities near zero, or compliance upstream of a merge point. Shoulders SH are systematic errors in the gradient, and means to minimize these errors are disclosed in co-pending, commonly owned U.S. Provisional Application entitled MICROFLUIDIC SYSTEMS, DEVICES AND METHODS FOR REDUCING DIFFUSION AND COMPLIANCE EFFECTS AT A FLUID MIXING REGION, U.S. Provisional Application No. 60/707,220 (Attorney Docket No. 447/99/3/1); and MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2), the contents of which are incorporated in their entireties.

Sample processing apparatus SPA can be useful for a wide variety of applications, due at least in part to the simplicity of the technique for concentration gradient mixing described hereinabove and the ubiquity of concentration gradients in assays. Non-limiting examples of applications include enzyme kinetics, clinical diagnostics for neo-natal care (e.g., blood enzyme diagnostics with microliter samples), toxicity studies for drug development (e.g., P450 assays or S9 fraction assays), flow cytometry, cell-based assays, and gradient elution for mass spectrometry.

In some embodiments, sample processing apparatus SPA provides for characterizing biochemical reactions. In some embodiments, characterizing the biochemical reaction comprises determining:

(1) steady-state kinetic constants, such as Michaelis constants for substrates (Kₘ), maximum velocity (Vₘₐₓ), and the resultant specificity constant (Vₘₐₓ/Kₘ or k_{cat}/Kₘ);

(2) binding constants for ligands (K_{d}) and capacity of receptor binding (Bₘₐₓ);

(3) kinetic mechanisms of a bi- or multi-substrate enzyme reactions;

(4) effect of buffer components, such as salts, metals and any inorganic/organic solvents and solutes on enzyme activity and receptor binding;

(5) kinetic isotope effect on enzyme catalyzed reactions;

(6) effect of pH on enzyme catalysis and binding;

(7) dose-responses of inhibitors or activators on enzyme or receptor activity (IC₅₀ and EC₅₀ values);

(8) mechanisms of inhibition of enzyme catalyzed reactions and associated inhibition constants (slope inhibition constant (Kᵢₛ) and intercept inhibition constant (Kᵢᵢ));

(9) binding constants (K_{d});

(10) binding stoichiometry; or

(11) combinations thereof.

The amount of data points and accuracy of collection for the above noted exemplary applications, when performed using the sample processing apparatus SPA described herein, are superior to that observed in any heretofore known data collection techniques. In particular, the sample processing apparatus SPA provides directly measurable continuous concentration gradients by accurately varying the volumetric flow rates of multiple reagent streams simultaneously by a precisely known amount. Therefore, it is known by direct observation what the expected concentration gradients are, rather than having to calculate the gradients indirectly. Alternatively, an inert “tracer” dye can be added to one of the reagent streams, and the concentration of this tracer dye measured downstream such that the concentration of the tracer dye reports the proportion of the reagent stream, including any noise or dispersion in the system This allows for more accurate data collection than is possible with previously described devices for the applications listed above and others. The pump mechanisms described herein facilitate the use of continuous concentration gradients, in that in one embodiment, the pump mechanisms operate by flow displacement, which provides more precise volume control.

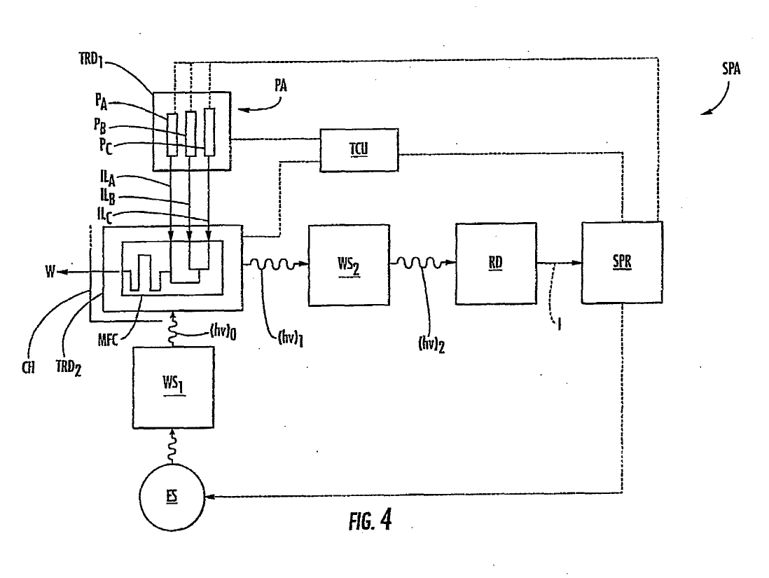

Referring now to FIG. 4, a generalized schematic of sample processing apparatus SPA is illustrated to show by way of example the integration of other useful components for analytical testing and data acquisition according to spectroscopic, spectrographic, spectrometric, or spectrophotometric techniques, and particularly UV or visible molecular absorption spectroscopy and molecular luminescence spectrometry (including fluorescence, phosphorescence, and chemiluminescence). In addition to pump assembly PA and microfluidic chip MFC, which at detection point DP (FIG. 1) could be considered as serving as a data encoding or analytical signal generating virtual sample cell or cuvette, sample processing apparatus SPA can include an excitation source ES, one or more wavelength selectors WS₁ and WS₂ or similar devices, a radiation detector RD, and a signal processing and readout device SPR. The particular types of these components and their inclusion with sample processing apparatus SPA can depend on, for example, the type of measurement to be made and the type of analytes to be measured/detected. In some embodiments, sample processing apparatus SPA additionally comprises a thermal control unit or circuitry TCU that communicates with a pump temperature regulating device TRD₁ integrated with pump assembly PA for regulating the temperature of the reagents residing in pumps P_{A}-P_{C}, and/or a chip temperature regulating device TRD₂ in which microfluidic chip MFC can be enclosed for regulating the temperature of reagents and mixtures flowing therein. Details of these temperature regulating components according to specific embodiments are provided in co-pending, commonly owned U.S. Provisional Application entitled MICROFLUIDIC BASED APPARATUS AND METHOD FOR THERMAL REGULATION AND NOISE REDUCTION, U.S. Provisional Application No. 60/707,330 (Attorney Docket No. 447/99/2/3 and U.S. Provisional Application entitled APPARATUS AND METHOD FOR HANDLING FLUIDS AT NANO-SCALE RATES, U.S. Provisional Application No. 60/707,421 (Attorney Docket No. 447/99/2/2), the contents of which are incorporated herein in their entirety. Additionally, a chip holder CH can be provided as a platform for mounting and positioning microfluidic chip MFC, with repeatable precision if desired, especially one that is positionally adjustable to allow the user to view selected regions of microfluidic chip MFC and/or align microfluidic chip MFC (e.g., detection point DP thereof) with associated optics.

Generally, excitation source ES can be any suitable continuum or line source or combination of sources for providing a continuous or pulsed input of initial electromagnetic energy (hv)₀ to detection area or point DP (FIG. 1) of microfluidic chip MFC. Non-limiting examples can include lasers, such as visible light lasers including green HeNe lasers, red diode lasers, and frequency-doubled Nd:YAG lasers or diode pumped solid state (DPSS) lasers (532 nm); hollow cathode lamps; deuterium, helium, xenon, mercury and argon arc lamps; xenon flash lamps; quartz halogen filament lamps; and tungsten filament lamps. Broad wavelength emitting light sources can include a wavelength selector WS₁ as appropriate for the analytical technique being implemented, which can comprise one or more filters or monochromators that isolate a restricted region of the electromagnetic spectrum. Upon irradiation of the sample at detection point DP, a responsive analytical signal having an attenuated or modulated energy (hv)₁ is emitted from microfluidic chip MFC and received by radiation detector RD. Any suitable light-guiding technology can be used to direct the electromagnetic energy from excitation source ES, through microfluidic chip MFC, and to the remaining components of the measurement instrumentation. In some embodiments, optical fibers are employed. The interfacing of optical fibers with microfluidic chip MFC according to advantageous embodiments is disclosed in a co-pending, commonly owned U.S. Provisional Application entitled MICROFLUIDIC CHIP APPARATUSES, SYSTEMS, AND METHODS HAVING FLUIDIC AND FIBER OPTIC INTERCONNECTIONS, U.S. Provisional Application No. 60/707,246 (Attorney Docket No. 447/99/4/2), the content of which is incorporated herein in its entirety. In some embodiments, a miniaturized dip probe can be employed at detection point DP, in which both the optical sending and returning fibers enter the same side of microfluidic chip MFC and a reflective element routes the optical signal down the sending fiber back through the microfluidic channel to the returning fiber. Similarly a single fiber can be used both to introduce the light and to collect the optical signal and return it to a detector. For example, the excitation light for a fluorophore can be introduced into the microfluidic chip by an optical fiber, and the fluorescent light emitted by the sample in the microfluidic chip can be collected by that same fiber and transmitted to a photodetector, with appropriate wavelength selectors permitting rejection of excitation light at the photodetector.

Wavelength selector WS₂ is utilized as appropriate for the analytical technique being implemented, and can comprise one or more filters or monochromators that isolate a restricted region of the electromagnetic spectrum and provide a filtered signal (hv)₂ for subsequent processing. Radiation detector RD can be any appropriate photoelectric transducer that converts the radiant energy of filtered analytical signal (hv)₂ into an electrical signal I suitable for use by signal processing and readout device SPR. Non-limiting examples can include photocells, photomultiplier tubes (PMTs), avalanche photodiodes (APDs), photodiode arrays (PDAs), and charge-coupled devices (CCDs). In particular, for fluorescence measurements, a PMT or APD can be operated in a photon counting mode to increase sensitivity or yield improved signal-to-noise ratios. Advantageously, radiation detector RD is enclosed in an insulated and opaque box to guard against thermal fluctuations in the ambient environment and keep out light.

Signal processing and readout device SPR can perform a number of different functions as necessary to condition the electrical signal for display in a human-readable form, such as amplification (i.e., multiplication of the signal by a constant greater than unity), phase shifting, logarithmic amplification, ratioing, attenuation (i.e., multiplication of the signal by a constant smaller than unity), integration, differentiation, addition, subtraction, exponential increase, conversion to AC, rectification to DC, comparison of the transduced signal with one from a standard source, and/or transformation of the electrical signal from a current to a voltage (or the converse of this operation). In addition, signal processing and readout device SPR can perform any suitable readout function for displaying the transduced and processed signal, and thus can include a moving-coil meter, a strip-chart recorder, a digital display unit such as a digital voltmeter or CRT terminal, a printer, or a similarly related device. Finally, signal processing and readout device SPR can control one or more other components of sample processing apparatus SPA as necessary to automate the mixing, sampling/measurement, and/or temperature regulation processes of the methods disclosed herein. For instance, signal processing and readout device SPR can be placed in communication with excitation source ES, pumps P_{A}-P_{C} and thermal control unit TCU via suitable electrical lines to control and synchronize their respective operations, as well as receive feedback from the encoders typically provided with pumps P_{A}-P_{C}.

As appreciated by persons skilled in the art, the signal processing, readout, and system control functions can be implemented in individual devices or integrated into a single device, and can be implemented using hardware (e.g., a PC computer), firmware (e.g., application-specific chips), software, or combinations thereof. The computer can be a general-purpose computer that includes a memory for storing computer program instructions for carrying out processing and control operations. The computer can also include a disk drive, a compact disk drive, or other suitable component for reading instructions contained on a computer-readable medium for carrying out such operations. In addition to output peripherals such as a display and printer, the computer can contain input peripherals such as a mouse, keyboard, barcode scanner, light pen, or other suitable component known to persons skilled in the art for enabling a user to input information into the computer.

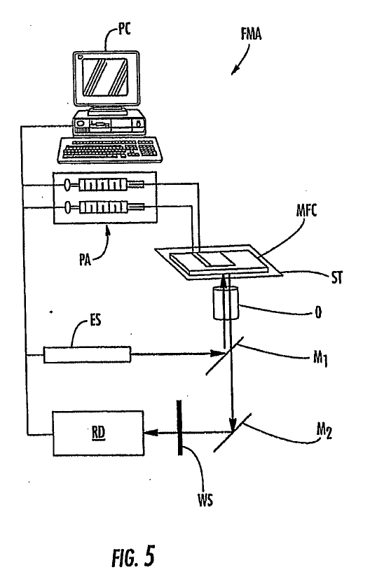

Referring now to FIG. 5, a specific embodiment of sample processing apparatus SPA is illustrated in the form of a fluorescence measurement apparatus, generally designated FMA, which can be used to measure/detect fluorescence intensity, polarization fluorescence, Fluorescence Resonance Energy Transfer (FRET), fluorescence lifetime, or combinations thereof. A microscope, and particularly a fluorescence microscope, can be employed for a number of functions. Microfluidic chip MFC can be mounted on a microscope stage ST typically provided with the microscope. In some embodiments, microscope stage ST can be controllably actuated in X-Y or X-Y-Z space to align microfluidic chip MFC with an objective O of the microscope as well as other associated optics, including confocal optics which permit analysis at a chosen location in three-dimensional space. In addition to enabling a selected area of microfluidic chip MFC to be viewed, objective O can focus or direct incoming light supplied from excitation source ES. Light-guiding optical components can be employed, including a dichroic mirror M₁ for reflecting the light from excitation source ES and transmitting the fluorescence signal from microfluidic chip MFC, and an additional mirror M₂ if needed for reflecting the attenuated signal to wavelength selector WS.

Fluorescence measuring apparatus FMA can be configured such that multiple excitation wavelengths are simultaneously introduced into a sample containing multiple signal fluorophores inside microfluidic chip MFC. This can be done by using a multiple bandpass filter as a wavelength selector WS₁ or by using multiple lasers as excitation light sources. Similarly multiple bandpass dichroic mirrors and multiple wavelength selectors WS₂ can be used to transmit the fluorescence from individual fluorophores to multiple signal processing and readout devices SPR.

In the embodiment illustrated in FIG. 5, mirror M₁ can be a longpass dichroic reflector that reflects light from excitation source ES and transmits fluorescent light collected from microfluidic chip MFC by objective O back toward radiation detector RD. Wavelength selector WS can be a barrier filter appropriate for use in conjunction with a radiation detector RD provided in the form of a photon counter. As further illustrated in FIG. 5, the signal processing and readout device SPR can be provided in the form of any suitable computer PC. A suitable computer program, developed for instance using LABVIEW® software, available from National Instruments Corporation, Austin, Tex., can be stored and/or loaded into computer PC to enable computer PC to be specifically programmed to control the operation of fluorescence measurement apparatus FMA.

As described above, sample processing apparatus SPA is useful for characterizing biochemical reactions on a microscale level, including for example determining potency of inhibitors (IC₅₀). In a typical IC₅₀ experiment, and with reference to FIG. 1, the reagent streams are combined to create a final reaction mix in a mixing channel MC₂. This reaction mix can then be advanced by the combined flows of pumps P_{A}, P_{B} and P_{C} into serpentine analysis channel SAC. As the reaction mix flows through aging loop SAC, the reaction proceeds and a reaction product can be measured at detection point DP. As described hereinabove, the flow rates of pumps P_{A}, P_{B}, and P_{C} can be controlled to produce a continuously variable concentration gradient of one or more reaction reagents such that as one pump decreases its flow rate, another pump increases its flow rate, such that the combined flow of the three pumps is held constant. Pump P_{A} can hold buffer, the inhibitory compound under test (the “inhibitor”), and a tracer dye that is used to report the concentration of the inhibitor. Pump P_{B} can contain buffer only. The reagent streams from pumps P_{A} and P_{B} can be run as a complementary pair, with their combined flow rates equaling, for example, 15 nL/min. Thus, the reagent streams from these two pumps combines at mixing point MP1. Pump P_{A} starts at 15 nL/min and pump P_{B} starts at 0 nL/min. After 2-3 minutes, the flow rate of pump P_{A} can be decreased linearly with time to 0 nL/min, and the flow rate of pump P_{B} increased linearly to 15 nL/min. The flow rate is then held at this flow rate for another 2-3 minutes. Thus, the combined flows of pumps P_{A} and P_{B} create a concentration gradient of the inhibitor and associated tracer dye, as is generally illustrated in FIG. 3B. The combined flows of pumps P_{A} and P_{B} flow from mix point MP₁ to mix point MP₂ where they combine with the flow of pump P_{C}. The reagent stream from pump P_{C} contains the enzyme or other biomolecule against which the inhibitor is being tested. The flow rate of pump P_{C} is constant at, for example, 15 nL/min such that the combined flow rates of pumps P_{A}, P_{B} and P_{C} is constant at 30 nL/min. Thus, the concentration of the enzyme is held constant, and the concentration of the inhibitor varies in the reagent stream.

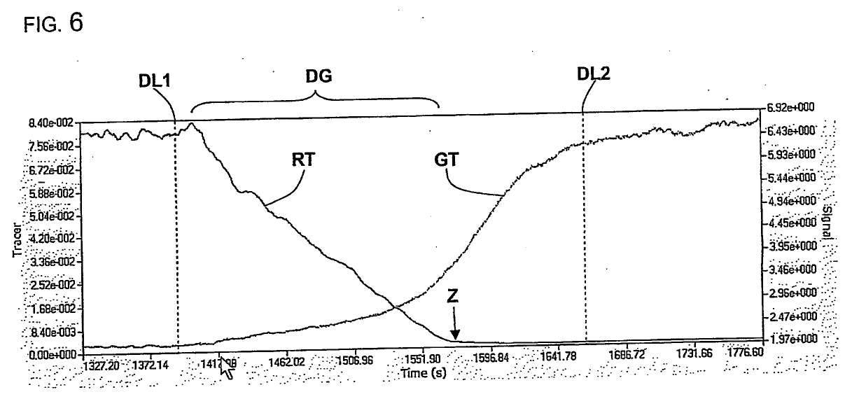

FIG. 6 shows an exemplary experiment to measure IC₅₀ using a sample processing apparatus SPA as depicted in FIG. 1. The pumps can contain the following:

P_{A}: inhibitor+tracer dye (e.g., ALEXA FLUOR 700™, Invitrogen, Carlsbad, Calif., U.S.A.)+enzyme substrate+buffer

P_{B}: enzyme substrate+buffer

P_{C}: coupling enzymes+target enzyme+AMPLEX RED® (10-acetyl-3,7-dihydroxyphenoxazine, from Invitrogen),

AMPLEX RED® is a non-fluorescent precursor that is converted to highly fluorescent resorufin by the action of the target enzyme and the coupling enzymes. The pump flow rates can be varied as follows:

| | 1300 to | 1380 to | 1560 to |
|---|---|---|---|
| | 1380 | 1560 | 1780 |
| Pump | seconds | seconds | seconds |
| P_{A} | 15 nl/min | Decrease to | 0 nl/min |
| | | 0 nl/min | |
| P_{B} | 0 nl/min | Increase to | 15 nl/min |
| | | 15 nl/min | |
| P_{C} | 15 nl/min | 15 nl/min | 15 nl/min |

Again, it is the complimentary actions of pumps P_{A} and P_{B} that create the concentration gradient of inhibitor and tracer dye at mixing point 1 MP₁ which then travels to mixing point 2 MP₂ where it is combined with the target enzyme.

Considering again FIG. 6, red trace RT is the fluorescence measured from the tracer dye (ALEXA FLUOR 700™) that was mixed with the inhibitor, so the concentration of the inhibitor should mirror the concentration of the tracer dye. Green trace GT is the fluorescence measured from a product (resorufin) of the coupled enzyme system, so it indicates the activity of the target enzyme. Dashed lines DL1 and DL2 delineate the region of data that is used for determining the IC₅₀ of the inhibitor. Thus, in this example, the concentration of the inhibitor is initially high and decreases to zero over the region labeled as a declining gradient DG. It can be seen from the graph that the activity of the enzyme increases, as evident by the rise in green trace GT; however, the activity continues to rise, even after the tracer dye reaches approximately zero at a point Z. In fact, green trace GT continues to rise until the end of the experiment at 1780 seconds. This continuing rise in green trace GT indicates that the concentration of the inhibitor continues to decrease even after tracer dye TD has reached zero.

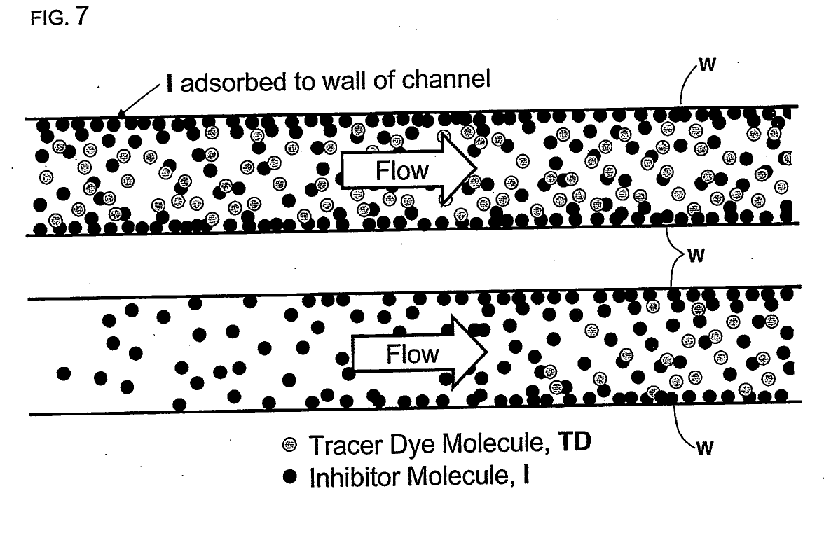

FIG. 7 illustrates an explanation for the results observed in this exemplary experiment and illustrated in FIG. 6. Reversible adsorption of an inhibitor I to one or more walls W of the microfluidic channels in microfluidic chip MFC as illustrated in FIG. 7 can cause the observed experimental results.

When the concentration of inhibitor I and its associated tracer dye TD in solution are high, inhibitor I can adsorb to walls W. As the concentration of inhibitor I decreases in the declining region of the concentration gradient (the molecules TD and I are swept to the right in FIG. 7), inhibitor I will desorb from the wall W. The result is that the concentration of inhibitor I free in solution will be higher than mixed at mixing point MP1 and, therefore, higher than reported by tracer dye TD. In fact, when the concentration of tracer dye TD has declined to zero (compare to point Z in FIG. 6), indicating the concentration of inhibitor I should also be zero, the concentration of the inhibitor I remains above zero until all inhibitor I has desorbed from the wall. Again, this is seen in FIG. 6 as the green trace GT continues to increase after point Z.

Adsorption of hydrophobic molecules can occur when the surface of a microfluidic channel also is hydrophobic. Microfluidic chips can be made from many materials, such as polymers, which can have hydrophobic surfaces. Many plastics have surface energies of 30 to 40 dyne/cm (more hydrophilic surfaces have higher surface energies), whereas water has a surface energy of about 73 dyne/cm. Many molecules measured in drug discovery have a clogP greater than about 4 indicating that they are highly hydrophobic. ClogP is a calculated version of “log P”, which is the log of the partition coefficient (water to octanol) for a molecule. LogP can be considered a measure of a molecule's hydrophobicity, with higher logP indicating greater hydrophobicity. Thus, it can be anticipated that hydrophobic binding of these compounds to a plastic surface can result in many molecules of interest to the drug industry showing behavior similar to FIGS. 6 and 7.

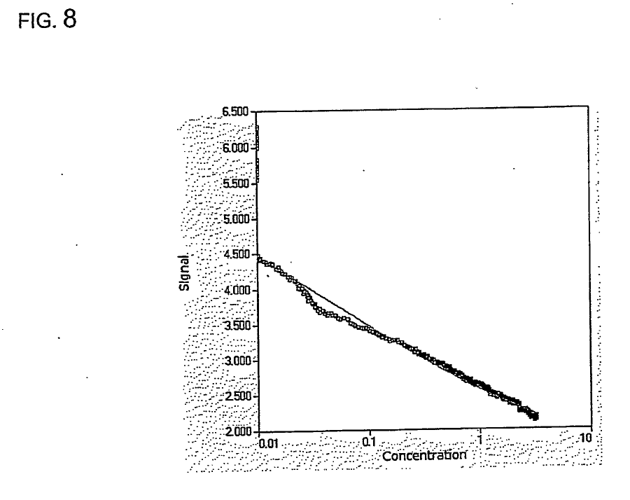

FIG. 8 presents the data from FIG. 6 transformed to concentration versus enzyme activity—the format which can be used to determine an inhibitor's IC₅₀. The x-axis is the concentration of the inhibitor, as reported by the tracer dye, which is red trace RT in FIG. 6. The y-axis is the enzyme activity as reported by green trace GT in FIG. 6. The data at inhibitor concentrations of 0.01 and below are rendered meaningless due to molecule adsorption, and as such this is the lowest concentration that can be measured from tracer dye TD. However, IC₅₀ for this inhibitor is determined from data falling within this range. Thus, the IC₅₀ of this inhibitor likely cannot be accurately determined from this data.

As shown in FIG. 7, inhibitor I can adsorb to channel walls W when at high concentrations as it flows through channels. Then, as shown in the lower drawing of FIG. 7, inhibitor I can desorb as its concentration drops. This phenomenon can account for the data reported in the graphs of FIGS. 6 and 7. Hydrophobic molecules in aqueous solutions adsorb to hydrophobic surfaces. Thus, the concentration of the compound in a volume of fluid decreases as it flows over a “clean surface” and, similarly, a later volume with no compound in it will have compound added as it flows past the surface and the compound desorbs. The effect of adsorption/desorption can be that the concentration of the molecule in the volume is no longer known. Such phenomena have been modeled in several reports (see e.g., Balasubramanian et al., 1997).

The presently disclosed subject matter provides apparatuses and methods for using the same that can decrease the interference of adsorption to concentration dependent measurements, such as in a biochemistry reaction including IC₅₀ determinations, by altering the geometry of a microfluidic channel. Although adsorption may not be eliminated, the change in concentration caused by adsorption can be minimized. In general terms, the effects of adsorption on measurements can be minimized by reducing the channel surface area to fluid volume within the channel ratio (S/V). This both decreases the amount of adsorption to the surface and increases diffusion distances, as described herein below. However, as a high surface area to volume ratio can be an unavoidable consequence of the miniaturization of microfluidics, the geometries provided by some embodiments of the presently disclosed subject matter to minimize adsorption consequences are most unexpected by persons in the field of microfluidics. The presently disclosed subject matter provides for, in some embodiments, using large channel diameters in regions of the microfluidic chip most affected by adsorption of reaction components, that is, in regions where measurements are taken. In some embodiments of the presently disclosed subject matter, and with reference to the microfluidic chip embodiment shown in FIG. 1, large channel diameters at detection point DP can be provided to reduce adsorption effects, as a substitute for or in combination with serpentine analysis channel SAC.

As such, the presently disclosed subject matter can provide in some embodiments a microfluidic analysis channel that can comprise an inlet having a first cross-sectional area for passage of fluid therethrough and an analysis region in fluid communication with the inlet and having a second cross-sectional area for passage of fluid from the inlet to the analysis region. The second cross-sectional area can be greater than the first cross-sectional area whereby adsorption of a compound in fluid in the analysis region is decreased and whereby reduction of concentration of the compound at a center axis region in the analysis region is minimized. In some embodiments, the second cross-sectional area can be at least two times larger than the first cross-sectional area. Further, in some embodiments, the second cross-sectional area can be between about two times and five hundred times larger than the first cross-sectional area. The analysis channel can be in fluid communication with one or more upstream microfluidic channels, as for example, the analysis channel can be in direct fluid communication with the upstream mixing channel MC₂, which in turn can provide fluid communication with the remaining microfluidic channels on microfluidic chip MFC. In some embodiments, the analysis channel has a surface area to volume ratio value of about 1/10^{th} or less a surface area to volume ratio value of an upstream microscale channel, such as for example mixing channel MC2. The analysis channel in some embodiments can comprise an expansion region beginning at the inlet and having an upstream cross-sectional area approximately equivalent to the inlet first cross-sectional area and a downstream cross-sectional area approximately equivalent to the second cross-sectional area. In some embodiments, the downstream cross-sectional area can be between about two times and about five hundred times larger than the upstream cross-sectional area. Further, in some embodiments, the analysis region can comprise a detection area located along at least a portion of a center axis region of the analysis region.

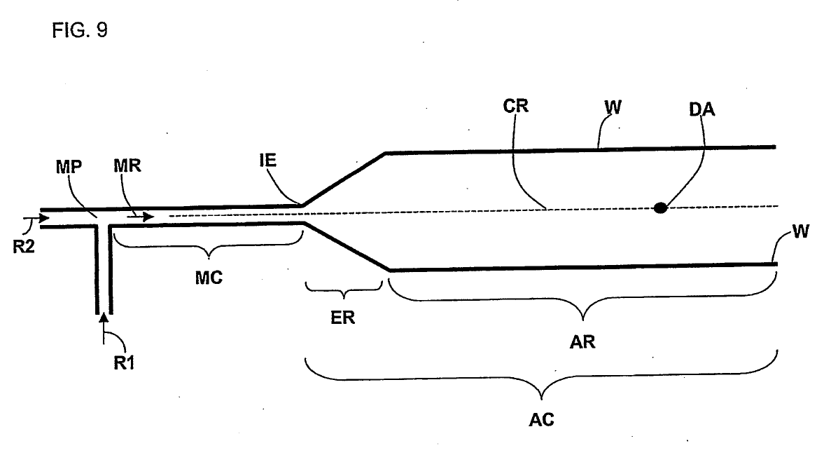

Turning now to FIG. 9, an embodiment of a novel analysis channel of the presently disclosed subject matter is illustrated in a top view. FIG. 9 shows the direction of flow represented by arrows R1 and R2 of two fluid reagent streams, which can combine at a merge region or mixing point MP. After combining into a merged fluid stream, the reagents within the stream can flow in a direction indicated by arrow MR down a mixing channel MC that can be narrow to permit rapid diffusional mixing of the reagent streams, thereby creating a merged fluid reagent stream. Mixing channel MC can have a length such that fluid at a particular flow rate resides a sufficient time in mixing channel MC to permit diffusional mixing of the reagents in the combined fluid streams. The fluid stream of reagents can then pass into an analysis channel AC, at an inlet or inlet end IE that can have a channel diameter and a cross-sectional area equivalent to that of mixing channel MC. The merged fluid stream can then flow through an expansion region ER that can have a cross-sectional area that can gradually increase and where the surface area to volume ratio can gradually decrease. The merged fluid stream can then continue into an analysis region AR of analysis channel AC with an enlarged cross-sectional area and a reduced surface area to volume ratio. A reaction can occur in analysis region AR of analysis channel AC, and measurements can be made inside this channel, such as with confocal optics, which as the term is used herein also refers to “quasi-confocal optics”, that is, optics that improve rejection of out-of-focus light by placing a small photodetector (smaller than the full image) in the image plane of the optical system, to achieve measurements at a detection area DA. Detection area DA can be located at a center axis region CR of analysis region AR of analysis channel AC. Center axis region CR can be a region equidistant from any channel wall W of analysis channel AC. Thus, the fluid at center axis region CR of detection area DA can be effectively “insulated” from adsorption at channel walls W. That is, the amount of any reagents removed at channel wall W can be too small, due to the greatly decreased surface area, and the diffusion distance to channel wall W can be too long, due to the greatly increased diffusion distance from center axis region CR to channel wall W, to greatly affect the concentration at center axis region CR. Confocal optics, for example, can reject signal from nearer channel wall W of analysis region AR, permitting measurements to be made at center axis region CR, or even more specifically at a center axis line, where the concentration is least affected by adsorption at channel wall W.

A consequence of increasing analysis channel AC cross-section by increasing channel diameter is that the ratio (S/V) of channel surface area to fluid volume within the channel is decreased, relative to a narrower channel. For example, to measure a reaction 3 minutes after mixing, with a volumetric flow rate of 30 nL/min, the reaction should be measured at a point in the channel such that microfluidic channel spanning from the mixing point MP to the detection area DA encloses 90 nL. For an analysis channel with square cross-section and a diameter of 25 μm, this point is about 144 mm downstream from mix point MP. This channel has a surface area of 1.44×10⁻⁵ square meters, yielding a surface to volume ratio S/V equal to 1.6×10⁵ m⁻¹. For a channel with a diameter of 250 μm, the measurement is made 1.44 mm downstream from mix point MP. This wider channel has a surface area of 1.44×10⁻⁶ square meters, yielding a S/V equal to 1.6×10⁴ m⁻¹, which is 1/10^{th} the S/V of the narrower channel. This alone can decrease ten-fold the removal of compound per unit volume by adsorption.

This geometry change can also decrease the radial diffusive flux of compound. Flow in these small channels is at low Reynolds number, so diffusion from a point in the fluid is the only mechanism by which compound concentration changes radially in a microfluidic channel. Increasing the radius of the channel, thereby decreasing the radial diffusive flux, therefore, means that the concentration of compound at center axis region CR of analysis region AR can be less affected by adsorption than in the smaller upstream channels.

Thus, increasing the cross-sectional area of analysis region AR of analysis channel AC can both decrease the amount of adsorption at the wall per unit volume and decrease the rate of flux of compound from center axis region CR to any of channel walls W. Both together mean that the concentration at center axis region CR can decrease more slowly due to adsorption of compound.

Further, in all embodiments, the surface area of all channels exposed to compounds, not just analysis channel AC, can preferably be kept minimal, especially those channels through which concentration gradients flow. This can be accomplished by making channels as short as practicable. Additionally, when the volume contained by a channel must be defined (e.g. where the channel must contain a volume of 50 nL), it is best to use larger diameters/shorter lengths wherever possible to reduce S/V.

Another consequence of increasing analysis channel AC cross-section by increasing channel diameter is that the length of the channel down which the fluid flows can be reduced. In the disclosure above, a channel with a 25 μm diameter was 144 mm long so as to enclose 90 nl whereas the channel with 250 μm diameter was only 1.44 mm long. This shorter channel can be much easier to fabricate and has a much smaller footprint on a microfluidic chip. An analysis channel having an increased cross-sectional area and decreased S/V as disclosed herein is simple to fabricate, acts passively without moving parts, and actually simplifies the design and fabrication of a microfluidic device because such an analysis channel measuring only a few millimeters in length can replace a serpentine analysis channel SAC as shown in FIG. 1 measuring many centimeters in length.

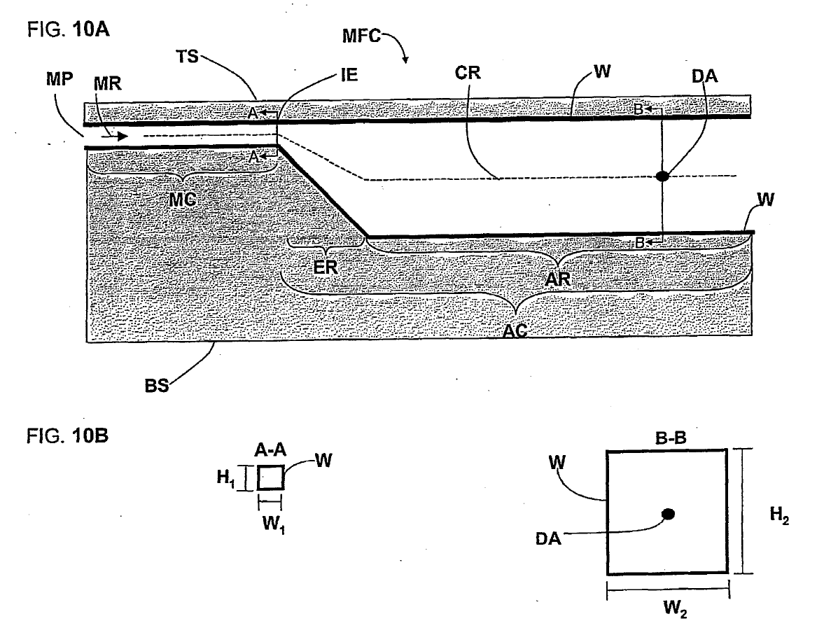

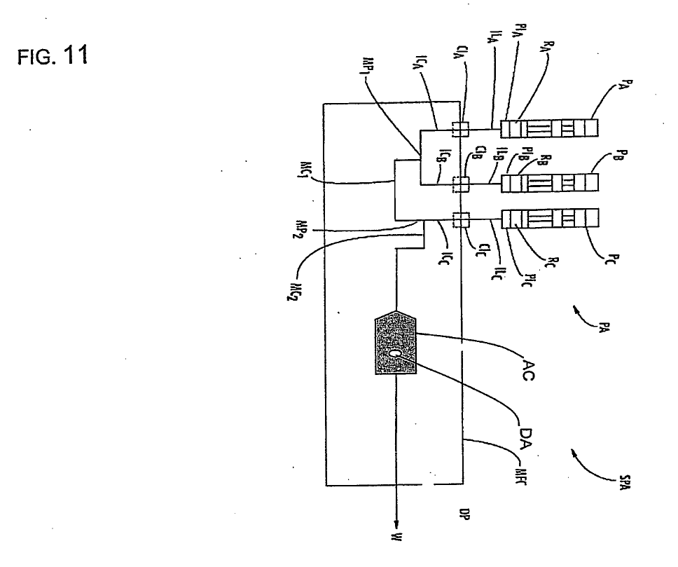

FIG. 10A presents a cross-sectional side view of a portion of a microfluidic chip MFC comprising mixing channel MC and analysis channel AC depicted in FIG. 9. Microfluidic chip MFC shown in FIG. 10A can be constructed by machining channels into a bottom substrate BS and enclosing channels by bonding a top substrate TS to bottom substrate BS or otherwise forming channels within microfluidic chip MC with bottom substrate BS and top substrate TS being integral. In FIG. 10A, only the flow of merged reagent fluid stream having a flow direction indicated by arrow MR after mixing point MP is shown. Flow in a microfluidic channel can be at low Reynolds number, so the streamline of fluid that flows along center axis region CR of the narrower mixing channel MC can travel at the mid-depth along entire mixing channel MC, becoming center axis region CR of analysis region AR of analysis channel AC. Detection area DA can reside along center axis region CR at a point sufficiently far downstream of mixing channel MC to permit the reaction to proceed to a desired degree.

Analysis channel AC can approximate a circular cross-section as closely as possible to produce the smallest ratio of surface area to volume, and also to produce the largest diffusion distance from center axis region CR to a channel wall W. However, microfluidic channels may not be circular in cross-section due to preferred manufacturing techniques. Rather, they can be more likely square in cross-section, with the exact shape depending on the technique used to form the channels. For such channels, a cross-section of analysis channel AC, particularly within analysis region AR, can have an aspect ratio as close to one as possible or, more precisely stated, the distance from center axis region CR to channel wall W can be as nearly constant in all radial directions as possible.

FIG. 10B shows two different cross-sectional views along analysis channel AC as viewed along cutlines A-A and B-B. Both cross-sectional views illustrate an aspect ratio approximating one. That is, for cross-section A-A, height H₁ of mixing channel MC is approximately equal to width W₁ of mixing channel MC, such that H₁/W₁ approximately equals one. Comparably, for cross-section B-B, height H₂ of mixing channel MC is approximately equal to width W₂ of mixing channel MC, such that H₂/W₂ approximately equals one.

FIG. 10B further shows that the cross-sectional area (H₂×W₂) of analysis region AR at cutline B-B, which is located at detection area DA of analysis region AR, is significantly larger than the cross-sectional area (H₁×W₁) of input end IE at cutline A-A. In some embodiments of the presently disclosed subject matter, the cross-sectional area at detection area DA can be at least twice the value of the cross-sectional area value at input end IE and further upstream, such as in mixing channel MC. Further, in some embodiments, the cross-sectional area at detection area DA can be between about two times and about five hundred times the value of the cross-sectional area value at input end IE. As shown in cutline B-B of FIG. 10B, detection area DA can be positioned along center axis region CR approximately equidistant from each of walls W to provide maximal distance from walls W, and thereby minimize effects of molecule adsorption to walls W. It is clear from FIG. 10B that the larger cross-sectional area at cutline B-B can provide both greater distance from walls W and smaller S/V than the smaller cross-sectional area at cutline A-A, both of which can reduce adsorption effects on data analysis, as described herein. Although detection area DA is shown in the figures as a circle having a distinct diameter, the depiction in the drawings is not intended as a limitation to the size, shape, and/or location of detection area DA within the enlarged cross-sectional area of analysis region AR. Rather, detection area DA can be as large as necessary and shaped as necessary (e.g. circular, elongated oval or rectangle, etc.) to acquire the desired data, while minimizing size as much as possible to avoid deleterious adsorption effects on the data. Determination of the optimal balance of size, shape and location while minimizing adsorption effects is within the capabilities of one of ordinary skill in the art without requiring undue experimentation.

FIGS. 6 and 8 present the results of an IC₅₀ measurement performed in a microfluidic chip MFC as depicted in FIG. 1, possessing a serpentine analysis channel SAC having a width of approximately 24 μm, a depth of approximately 22 μm and a length of approximately 25 cm. In microfluidic chip MFC (FIG. 1), adsorption prevents measurement of the inhibitor's IC₅₀ as illustrated in FIG. 6.

FIG. 11 depicts an alternative microfluidic chip MFC embodiment of the presently disclosed subject matter which can have an analysis channel AC with a much larger cross-sectional area and smaller S/V than the embodiment depicted in FIG. 1. The embodiment shown in FIG. 11 can share much of the structure of the signal processing apparatus embodiment shown in FIG. 1, except sample processing apparatus SPA of FIG. 11 can incorporate analysis channel AC having an enlarged cross-sectional area, rather than or in addition to serpentine analysis channel SAC.

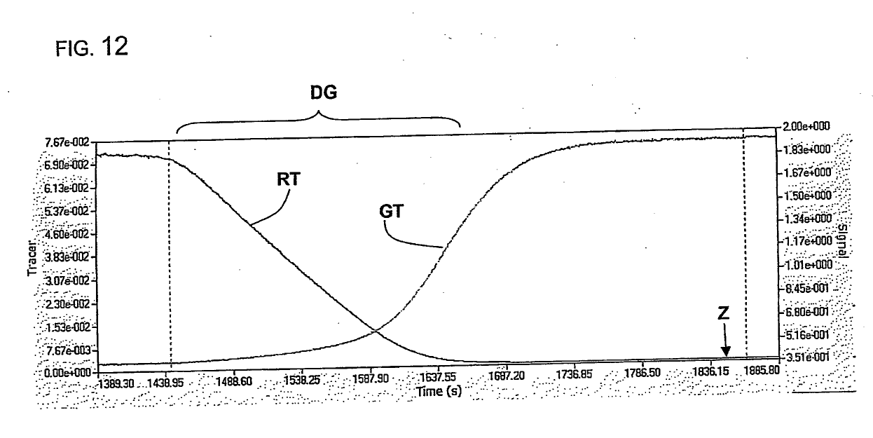

FIG. 12 is a graph showing data from an experiment identical to that described hereinabove for determining an IC₅₀ of an inhibitor and shown in FIG. 6, except FIG. 12 was performed in a microfluidic chip MFC as depicted in FIGS. 9-11 having an analysis channel AC measuring 190 μm wide and about 180 μm deep.

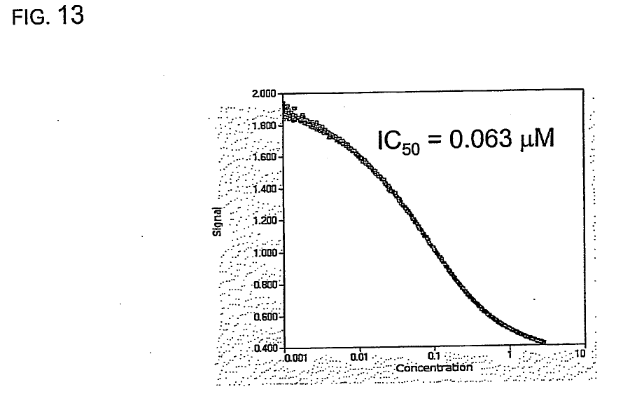

FIG. 13 presents the data from FIG. 12 transformed to concentration versus enzyme activity, which is comparable in presentation to the data shown in FIG. 8. In FIG. 13, the data at inhibitor concentrations below 0.01 μM are now accurate, unlike in FIG. 8, because now tracer dye TD and inhibitor I travel together. Consequently, the IC₅₀ of this inhibitor can be determined from this data. The measured value of 0.063 μM is very close to the IC₅₀ of 0.100 μM obtained from more conventional measurements in a microtiter plate.

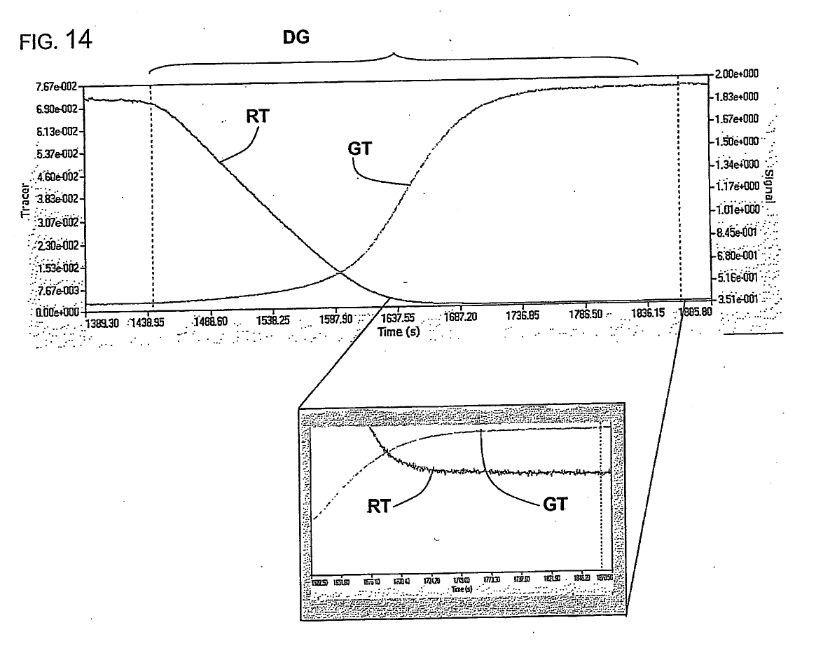

FIG. 14 presents another advantage of analysis channel AC having an increased cross-sectional area. The noise in both red trace RT and green trace GT is greatly reduced. FIG. 14 presents data collected using the same experimental procedure, reagents and apparatuses (except for the substitution of microfluidic chip MFC shown in FIG. 11 for microfluidic chip MFC shown in FIG. 1) as data shown in FIG. 12. The data of FIG. 14 were analyzed and presented as in FIG. 12, but now a magnified portion of the two traces is also presented (note that the offset for the two traces has been independently changed) showing the very low noise and, notably, that tracer dye TD concentration measured from red trace RT can be measured to a very low level. In the magnified portion, red trace RT discernibly decreases. This also permits accurate measurement of the low inhibitor concentrations needed to determine the IC₅₀ of the inhibitor, as shown in FIG. 13.

The reduced noise in measurements performed in analysis channel AC having increased cross-sectional area can result from the larger axial dispersion that occurs in analysis channel AC relative to the axial dispersion that occurs in the narrow serpentine analysis channel SAC. This larger axial dispersion causes noise in the concentration gradient to be dissipated, i.e. the noise in the concentration gradient is filtered out. The noise is a local fluctuation in the concentration gradient. Short regions of the gradient can be steeper than desired. Dispersion can cause concentration gradients to dissipate, with steeper gradients dissipating more rapidly. Thus, the locally steep regions of the gradient (i.e. the noise), can dissipate more rapidly than declining gradient DG overall.

The increased axial dispersion in analysis channel AC can in some instances create artifacts and systematic errors in certain measurements. For example, consider the gradient presented in FIGS. 6 and 12. These data were both generated by the same pumps containing the same solutions and executing the same flow rates. However, declining gradients DG in red traces RT measured at the detection points in the two analysis channels (serpentine analysis channel SAC in FIG. 6 and analysis channel AC in FIG. 12) are different. Although red trace RT in FIG. 12 has lower noise, it also has a different shape due to the axial dispersion in the channel.

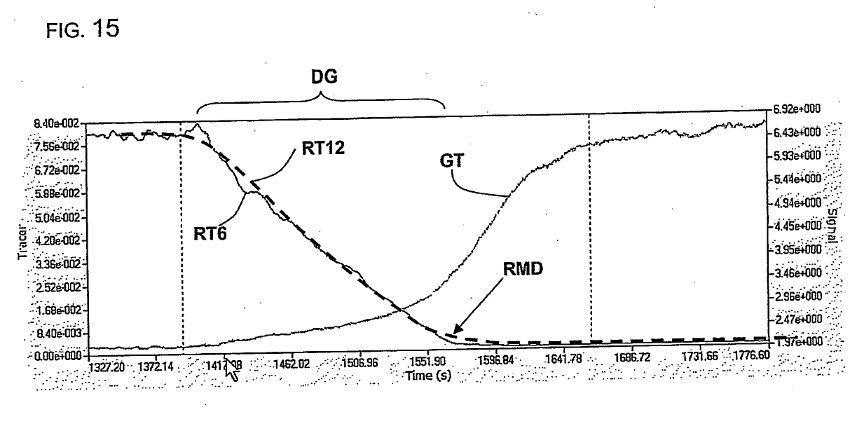

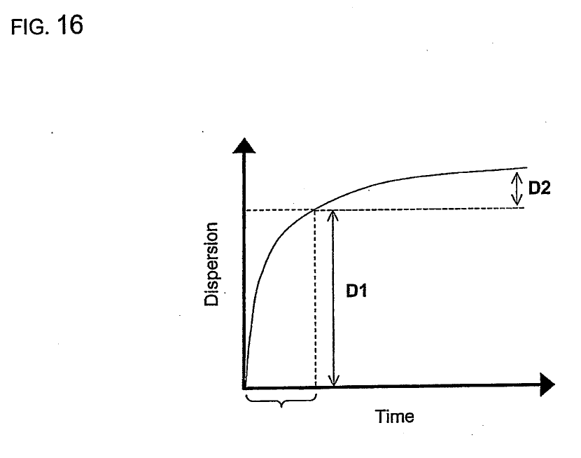

FIG. 15 presents a red trace RT6 identical to red trace RT from FIG. 6 and a red trace RT12 identical to red trace RT from FIG. 12 drawn as a black dashed line. The two traces do not agree perfectly, due to the increased dispersion experienced by the tracer dye and the inhibitor in analysis channel AC from the experiment sown in FIG. 12. The region of maximum difference RMD occurs at the “foot” of the gradient, where the linear decline of red trace from FIG. 6 RT6 transitions to a flat “zero” reading.

This region of maximum difference occurs where measurements often must be made for many inhibitors because an IC₅₀ determination can span 2-3 logs of concentration, and for a set of inhibitors with varying IC₅₀, many will have IC₅₀s near the bottom of the gradient, thus data from this area are often used for IC₅₀ determinations. Errors here can therefore be problematic.

One problem with the dispersion observed in the region of maximum difference RMD is that enzyme in this region of the fluid experiences a time-varying concentration of inhibitor. Consider a volume of fluid that travels from mixing point 2 MP₂ to detection area DA in microfluidic chip MFC depicted in FIG. 11. The gradient of the inhibitor, represented by the gradient of tracer dye TD, which is measured as red trace RT, initially starts as a linear declining gradient with a sharp transition as the flow of the pump containing inhibitor transitions to zero. As this volume of fluid enters analysis channel AC, tracer dye TD and inhibitor I disperse into analysis region AR from the higher concentrations upstream, causing the concentration of inhibitor I to rise as the volume travels down analysis channel AC. In this particular experiment, the enzyme molecules do not diffuse out or into this volume both because they have a much larger coefficient of diffusion (the enzyme has a larger molecular weight than the inhibitor) and because there is no concentration gradient of enzyme. Thus, the enzyme in this volume experiences an increasing concentration of inhibitor as it flows along analysis channel AC.

This time-varying concentration of inhibitor violates the assumptions of steady-state enzyme kinetics that underlie the mathematical methods used to calculate the inhibitor's IC₅₀ with regard to the enzyme. Thus, any determinations of the IC₅₀, or any other measurement assuming constant concentrations, from such a gradient could suffer from errors arising from dispersion.

This error can be reduced by the inclusion of a “controlled dispersion element” (CDE) in some embodiments between mixing point 1 MP1 and mixing point 2 MP2. A controlled dispersion element can take on a number of geometric forms that facilitate dispersion of the reagents combined at mixing point 1 MP1. Controlled dispersion elements according to some embodiments are described in more detail in co-pending, commonly owned U.S. Provisional Application entitled MICROFLUIDIC SYSTEMS, DEVICES AND METHODS FOR REDUCING NOISE GENERATED BY MECHANICAL INSTABILITIES, U.S. Provisional Application No. 60/707,245 (Attorney Docket No. 447/99/3/2), the content of which is incorporated herein in its entirety. In some embodiments, a CDE can be a serpentine channel or an expansion channel that increases dispersion of the gradient at a point in a microfluidic chip. In the exemplary experiments described herein, insertion of a CDE after mixing point 1 MP1 to thoroughly mix the inhibitor and substrate and before these reagents combine with enzyme at mixing point 2 MP2 in the microfluidic chip MFC can reduce the above described data errors due to dispersion. The result of such a controlled dispersion element is that the majority of the dispersion in the gradient occurs upstream of mixing point 2 MP2 before the gradient of inhibitor is mixed with the enzyme at mixing point 2 MP2, thereby preventing data errors due to improper dispersion of reagents prior to analysis at detection area DA.

Dispersion does not occur linearly with time. Consider FIG. 16, which shows the extent of dispersion of a gradient over time. Dispersion can be initially fast. However, dispersion causes the gradient to become less steep, which can cause dispersion to slow over time. If a controlled amount of dispersion D1 can be introduced into a gradient of inhibitor before it combines with the flow of enzyme, then the only dispersion, and its resulting time-varying concentration of inhibitor, that will be experienced by the enzyme is the much smaller dispersion D2.

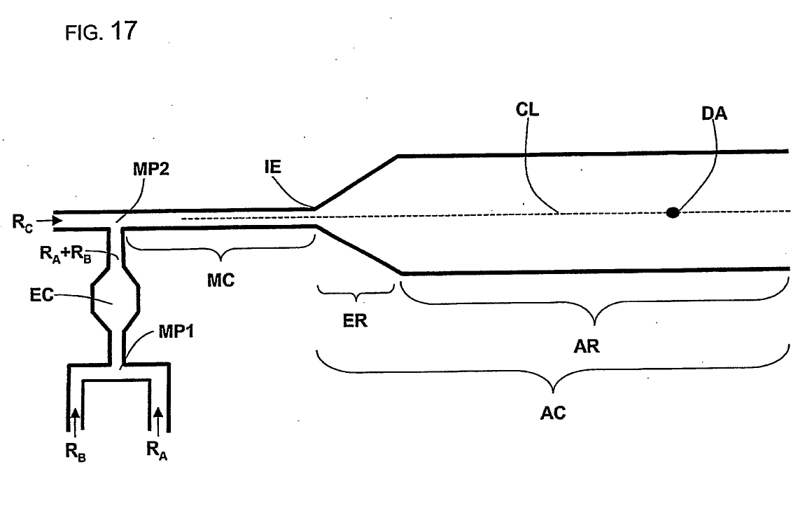

Now turning to FIG. 17, an embodiment of a microfluidic channel MFC having an analysis channel AC as depicted in FIG. 9, which can further include three reagent streams, whose flow direction is depicted as arrows R_{A}, R_{B}, and R_{C}, and each of which can be pumped by pumps P_{A}, P_{B} and P_{C}, respectively, as depicted in FIG. 11 is shown. Reagent streams whose flow direction is depicted as arrow R_{A} and R_{B} merge at mixing point 1 MP1, and combined stream R_{A}+R_{B} merges with a stream whose flow direction is shown as arrow R_{C} at a mixing point 2 MP2. As described for the experiments above, reagent stream R_{A} contains inhibitor I, and tracer dye TD, and buffer, while reagent stream R_{B} contains only buffer. Pumps P_{A} and P_{B} generating the flow of reagent streams R_{A} and R_{B}, are controlled to create a concentration gradient of inhibitor I and tracer dye TD at mixing point 1 MP1. This gradient flows through a CDE, which in this embodiment is shown as an expansion channel EC, that is, a channel having an enlarged diameter. Dispersion in expansion channel EC can be much larger than in a corresponding section of narrower channel, such as for example mixing channel MC owing to the larger diameter of the channel. Thus, a controlled amount of dispersion can be introduced into the gradient between mixing point 1 MP1 and mixing point 2 MP1. One of skill will appreciate that by engineering expansion channel with varying selected channel diameters and lengths, dispersion can be predictably controlled as desired. Thus, when combined stream R_{A}+R_{B} merges with stream R_{C} at mixing point 2 MP2, the majority of dispersion of the gradient has already transpired, and the target enzyme, which is in the R_{C} stream, only experiences changes in inhibitor concentration arising from the much smaller dispersion D2 (FIG. 16) that occurs after mixing point 2 MP2. Thus, introduction of a controlled dispersion element, such as for example expansion channel EC, between mixing point 1 MP1 and mixing point 2 MP2 can reduce errors in IC₅₀ measurements, or any other gradient-dependent measurement, arising from dispersion in an analysis channel, such as analysis channel AC. Similarly, a second controlled dispersion element can be used upstream of mixing point 2 MP2 for reagent stream R_{c} to reduce noise in reagent stream R_{c} if that stream is composed of two combined streams (e.g. R_{c}=R_{d}+Rₑ).

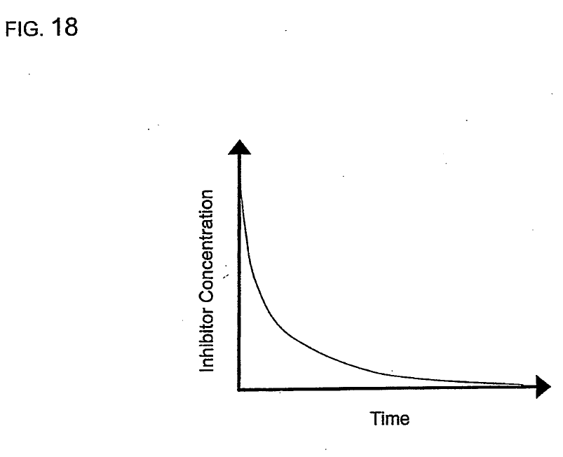

The error arising from the dispersion in analysis channel AC can also be minimized by generating a gradient that has no sharp transitions. Dispersion causes time-varying concentrations at sharp transitions, so elimination of these transitions minimizes time-varying concentrations. Thus, the pumps can generate a gradient that approximates a gradient that has already dispersed. For example, pumps P_{A} and P_{B} (FIG. 11) can be controlled to create an exponential gradient, as shown in FIG. 18.

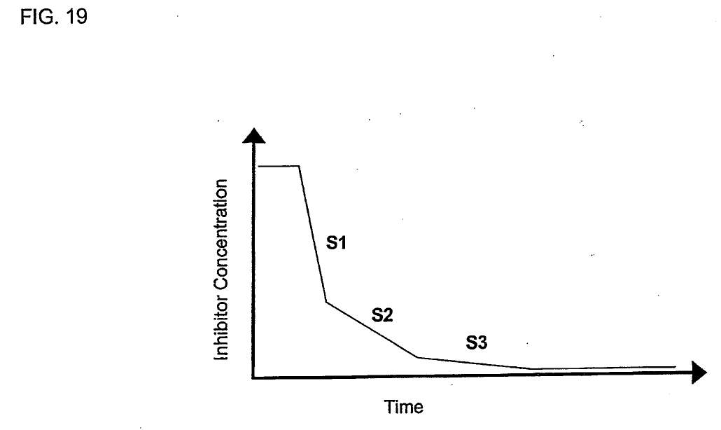

Other gradients can be used as well. For example, by creating a gradient with two or more slopes by controlling pump flow rates, sharp transitions can be reduced. FIG. 19 shows a gradient with three slopes S1, S2, and S3 that can reduce the magnitude of transitions in the gradient.

Pump-controlled gradients with reduced transitions and controlled dispersion elements can be combined to further reduce errors in measurements arising from dispersion.

The presently disclosed subject matter further provides methods for using the novel apparatuses described herein. In some embodiments, methods for decreasing adsorption of a compound in a fluid and minimizing reduction of concentration of the compound in a microfluidic device are provided.

In other embodiments of the presently disclosed subject matter, methods for making concentration dependent measurements in a microfluidic device are provided. In some embodiments, the method comprises flowing a fluid stream comprising at least one compound through at least one microscale channel of a microfluidic device, continuously varying the concentration of the compound within the fluid stream, flowing the fluid stream through an analysis channel in fluid communication with the microscale channel, and measuring the fluid stream at the detection area along at least a portion of the continuously varying concentration gradient of the molecule.

In some embodiments, the analysis channel is a novel analysis channel as disclosed herein and comprises an inlet having a first cross-sectional area for passage of the fluid stream therethrough, an analysis region in fluid communication with the inlet and having a second cross-sectional area for passage of the fluid stream from the inlet to the analysis region, the second cross-sectional area being greater than the first cross-sectional area, whereby adsorption of the compound in the fluid stream in the analysis region is decreased and a reduction of concentration of the compound at a center axis region in the analysis region is minimized, and a detection area located within the analysis region.

In some embodiments of the methods, analyzing a product in the analysis region comprises utilizing confocal optics so that data can be collected at a center axis region of the analysis channel, which provides data the least impacted by adsorption effects at the channel walls.

In some embodiments of the methods, analyzing a product or making concentration dependent measurements comprises determining steady-state kinetic constants; Michaelis constants (Kₘ), kinetic isotope effects on enzyme catalyzed reactions; dose-responses of inhibitors or activators on enzyme or receptor activity (IC₅₀ and EC₅₀ value); mechanisms of inhibition of an enzyme catalyzed reaction and associated inhibition constants (slope inhibition constant (Kᵢₛ) and intercept inhibition constant (Kᵢᵢ)); interaction factors between multiple inhibitors (α); kinetic mechanisms of multi-substrate enzyme reactions; capacity of receptor binding (Bₘₐₓ); pH effects on enzyme catalysis; pH effects on enzyme binding; binding constants (K_{d}); binding stoichiometry; or combinations thereof. Disclosure related to analyzing reaction products or making concentration dependent measurements is provided in U.S. Provisional Application entitled MICROFLUIDIC METHODS AND APPARATUSES FOR FLUID MIXING AND VALVING, U.S. Provisional Application No. 60/707,329 (Attorney Docket No. 447/99/2/4); U.S. Provisional Application entitled METHODS FOR CHARACTERIZING BIOLOGICAL MOLECULE MODULATORS, U.S. Provisional Application No. 60/707,328 (Attorney Docket No. 447/99/5/1); and U.S. Provisional Application entitled METHODS FOR MEASURING BIOCHEMICAL REACTIONS, U.S. Provisional Application No. 60/707,370 (Attorney Docket No. 447/99/5/2).

In some embodiments of the methods, an analyzed molecule is fluorescently labeled. Further, in some embodiments, measuring or analyzing the fluorescently labeled molecule comprises measuring fluorescence intensity, polarization fluorescence, fluorescence resonance energy transfer (FRET), fluorescence lifetime, or combinations thereof.

In some embodiments of the methods, continuous concentration gradients of reagents are generated by controlled variation of volumetric flow rates of pumps dispensing fluid streams comprising the reagents, as described in detail hereinabove. Further, in some embodiments, continuous concentration gradients of one or more reagents are created having multiple slopes (see, e.g. FIG. 19). Still further, in some embodiments, logarithmic continuous concentration gradients of one or more reagents are created.

In some embodiments, the presently disclosed subject matter further provides apparatuses and methods for making and using the same that can decrease the interference of adsorption to concentration dependent measurements, such as in biochemistry reactions (including IC₅₀ determinations), by reducing adsorption of molecules to microfluidic channel walls. In some embodiments, the presently disclosed subject matter provides microfluidic chips comprising channels and chambers with treated surfaces exhibiting reduced adsorption of molecules to channel walls, such as for example hydrophilic surfaces, and methods of preparing and using the same. In some embodiments, methods of preparing hydrophilic surfaces by treating hydrocarbon-based plastics, such as for example polycarbonate, with fluorine gas mixtures are provided. In some exemplary embodiments, the methods comprise contacting a mixture of fluorine gas and an inert gas with the surface to be treated, then flushing the surface with air. This treatment results in plastic surfaces of increased hydrophilicity (increased surface energy). Hydrophobic solutes, in particular known and potential drug compounds, in solutions in contact with these treated hydrophilic plastic surfaces are less likely to be adsorbed onto the more hydrophilic surfaces. Plastics comprising the treated surfaces are useful in providing many improved drug discovery and biochemical research devices for handling, storing, and testing solutions containing low concentrations of hydrophobic solutes.

Additional details and features of hydrophilic surfaces in microfluidic systems and methods of making and using the same are disclosed in co-pending, commonly owned U.S. Provisional Application entitled PLASTIC SURFACES AND APPARATUSES FOR REDUCED ADSORPTION OF SOLUTES AND METHODS OF PREPARING THE SAME, U.S. Provisional Application No. 60/707,288 (Attorney Docket No. 447/99/9).

Further, in some embodiments of the presently disclosed subject matter, microfluidic systems are provided comprising an analysis channel with an enlarged cross-sectional area and a reduced surface area to volume ratio and, further comprising channels and chambers with hydrophilic surfaces.

### EXEMPLARY DESCRIPTION

The following is a detailed derivation and description of underlying mathematics supporting the use of analysis channels having increased cross-sectional areas and reduced S/V ratios when compared to standard microscale channels to reduce the deleterious effects of molecule adsorption to walls within microscale channels.

Symbols used in this description and relevant dimensions:
A cross-sectional area of a channel, μm²
C concentration, moles·liter⁻¹
dC/dx concentration gradient
D diffusion coefficient, cm²·s⁻¹
D′ Taylor-Aris effective diffusion coefficient, cm²·s⁻¹
F diffusive flux, moles·s⁻¹
l length, μm
k ratio of two cross-sectional areas such that A₂=kA₁
r radius, μm
O volume, μm³
Q volumetric flow rate, nl/min
V the average velocity in a channel (not the axial velocity)
W area of the wall, μm²
Δ abbreviation for dC/dx
Φ 48π²
π pi
Ψ F₁/F₂, the ratio of fluxes at two different axial points in a channel

There are at least three options for reducing molecule adsorption or effects thereof to the inner walls of microfluidic channels: (1) stop the sticking with an appropriate universally “non-sticky” plastic or coating, (2) correct for sticking algorithmically, and (3) design of the channel geometry/architecture to reduce sticking and/or its effects. The presently disclosed subject matter addresses the problem through novel exploitation of option 3 as described herein in detail.

The phenomenon of adsorption is a convolution of the following phenomena:
axial diffusion and radial diffusion generated by the parabolic velocity profile (Taylor-Aris dispersion), wherein axial is the direction down the length of the channel and radial is perpendicular to the axis, that is the direction from the centerline or center axis region of the channel to the wall;
convection which is described by the parabolic velocity profile at low Re, and
sticking of compounds at the wall.

Most treatments of this phenomenon examine only axial diffusion and dispersion, using the equations of Taylor-Aris which define an effective diffusion, or “dispersion”, coefficient, D′. This is a single term that encompasses the effects of axial diffusion and radial diffusion (coupled to the velocity gradient) to describe the net flux (“dispersion”) axially. It does not examine how sticking at the wall creates a radial concentration gradient which causes diffusion from the volume of fluid to the wall. Rather, most treatments make the simplifying assumption that a volume of fluid in a channel instantaneously comes to equilibrium radially because radial diffusion distances are small.

This simplifying assumption is not valid in large diameter channels, such as in the novel analysis channels disclosed herein because the diffusion distance from centerline to wall becomes large such that the volume does not go to equilibrium. Thus, the strategy adopted herein is to examine those channels for which the assumption of instantaneous radial mixing is probably not valid (e.g. large diameter channels, such as for example the novel analysis channels disclosed herein) and then to try to determine if such geometries can reduce the effects of sticking. The advantage of this approach is that it will work even if sticking, or adsorption, occurs. The more specific strategy attempted with this description is to see if diffusive flux from the centerline of the flow to the wall can be reduced. Thus, adsorption will not affect data gathered in a large diameter analysis channel if the concentration at the centerline remains unaffected and if data gathering is restricted to an area near or at the centerline.

There are a few interacting phenomena to consider when channel diameters are changed. These are primarily driven by the phenomenon in fluid mechanics known as “continuity of flow”. This states that for the flow of an incompressible fluid in a non-compliant channel (e.g. water in microfluidic channels), the volumetric flow rate, Q, is constant at all points along the channel. Volumetric flow rate, e.g. nl/min, is given by:

Q=VA (Eq. 1)

where V is the average velocity in the channel, and A is the cross-sectional area of the channel. The effect of increasing the diameter is thus to increase A and to decrease V. This leads to a number of interacting complexities:
(1) The diffusion distance from centerline to wall is larger in wider channels, as described above, so diffusive flux is slower;
(2) the surface area of wall per volume of fluid (the ratio of surface area to volume) becomes smaller such that the wall represents a smaller relative sink of compound;
(3) continuity of flow drives several related changes in the shape of a “unit” volume of fluid with the following effects:
   a. axial concentration gradients become steeper in larger diameter channels.
   Using continuity, the gradient becomes larger as

Δ₂=(A₂/A₁)Δ₁, where Δ=dC/dx. (Eq. 0)
b. radial concentration gradients become shallower in larger diameter channels, so diffusive flux is slower (this is related to the diffusion distance described in (1)).
c. axial velocity becomes slower; and
(4) the velocity at the centerline V_{CL} (sometimes also known as Vₘₐₓ because the velocity is maximal at the centerline) becomes smaller, and the radial velocity gradient becomes shallower (dV/dr becomes smaller).

The remainder of this description will develop some simple equations that describe these phenomena such that scaling relationships can be achieved. The description does not attempt a coupled solution of all the interacting differential equations.

Expansion channels share some of the underlying physics of large diameter analysis channels described herein, with certain distinct differences. Expansion channels are intended to increase dispersion in a channel. Further, at the end of an expansion channel, the channel diameter is again constricted so that, once again, any radial concentration differences are quickly dissipated by diffusion, as herein described above.

The description hereinbelow directed to the novel large diameter analysis channels disclosed herein relates to examining what happens after an expansion, but without the subsequent constriction that occurs in an expansion channel. Thus, this description is for the purpose of determining if diffusive flux from the centerline of the flow to the wall can be reduced such that adsorption at the wall is of little consequence at the centerline where data are collected. One of skill in the art will appreciate that measurements at the centerline must be performed with confocal optics or equivalent technology, or measurements distant from the centerline, where adsorption will have a larger effect, will corrupt measurements.

Diffusion is at the heart of these phenomena, so one must bear in mind that fluids containing molecules are mixed/flowed with very different diffusion coefficients, D. Some examples values of D are:

| | | Diffusion Coefficient |
|---|---|---|
| Compound | Molecular Weight | (cm² · s⁻¹) |
| NaCl | NA | ~1.5e−5 |
| glycine | 75 | 9.33e−6 |
| sucrose | 342 | 4.59e−6 |
| lysozyme | 14,100 | 1.04e−6 |
| Bovine serum albumin | 66,500 | 6.01e−7 |
| Tropomyosin | 93,000 | 2.2e−7 |
| Fibrinogen | 330,000 | 1.98e−7 |
| Myosin | 493,000 | 1.10e−7 |

(source: C. R. Cantor and P. R. Schimmel. 1980. Biophysical Chemistry. Part II. Techniques for the Study of Biological Structure and Function. W. H. Freeman & Co.)

The diffusion equation referred to frequently hereinbelow is Fick's law, given by

$$F = DA \frac{d C}{d x} \qquad ( Eq . 2 )$$

where F is the diffusive flux (moles/s) in the direction perpendicular to the cross-sectional area, A, across which the diffusion occurs; D is the diffusion coefficient for the molecule in question, and dC/dx is the concentration gradient.

Taylor and Aris derived a description for an “effective” diffusion coefficient, D′, that allows calculation of the average axial flux in a flow where the velocity gradient from centerline to wall generates radial concentration gradients. This is

$${D}^{′} = D + \frac{{r}^{2} {V}^{2}}{48 D} \qquad ( Eq . 3 )$$

The flow considered with regard to the description herein is a transition to a channel with a different cross-sectional area. Specifically, we are comparing dispersion on transition from a small channel to a large channel. As equation 1 states, this will cause the average velocity V to change. It is best to restate equation 3 using volumetric flow rate Q by substituting equation 1 into equation 3. This gives:

$${D}^{′} = D + \frac{{Q}^{2}}{48 {π}^{2} {r}^{2} D} = D + \frac{{Q}^{2}}{48 π A D} \qquad ( Eq . 4 )$$

Again, this describes axial flux averaged over the area of the channel. The term on the left gives the contribution to D′ from the diffusion coefficient D. The term on the right gives the contribution to D′ from the parabolic velocity gradient. Basically, as the radius gets larger, D′ approaches D such that axial flux is dominated by diffusion. This is a somewhat counterintuitive result—the dispersion coefficient gets smaller as the channel gets wider. The typical form of Taylor-Aris dispersion (equation 3) states that dispersion increases with increasing radius. The result arises from the fact that the average velocity V decreases due to continuity as the radius r increases.

The point of equivalency, that is, the point where the left- and right-hand terms contributes equally, occurs where

$$D = \frac{{Q}^{2}}{48 {π}^{2} {r}^{2} D} \\ {r}^{2 =} \frac{{Q}^{2}}{48 {π}^{2} {D}^{2}} \\ r = \frac{Q}{π \sqrt{48} D} \qquad ( Eq . 5 )$$

Typical numbers for exemplary flows include:

Q=30 nl/min=(30×10⁻¹² m³/60 s)=5×10⁻¹³ m³/s

$$D = 3 \times {10}^{- 6} {cm}^{2} \text{/} s = 3 \times {10}^{- 10} {m}^{2} \text{/} s$$

$$r = \frac{Q}{π \sqrt{48} D} = \frac{5 \times {10}^{- 13}}{π \sqrt{48} ( 3 \times {10}^{- 10} )}$$

$$r = 76.5 µ m$$

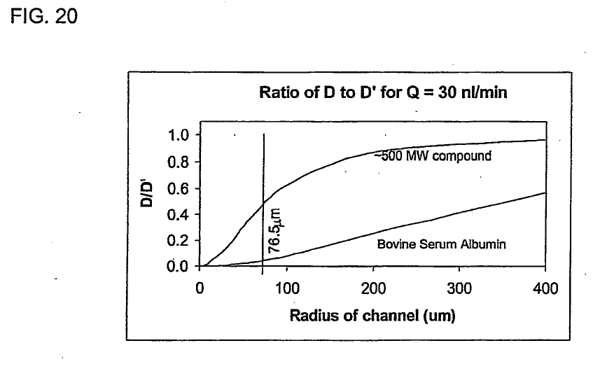

FIG. 20 plots the ratio D/D′ for channels of different diameters at a volumetric flow rate of 30 nl/min. (Note: D/D′=1 is not the point of equivalency given in Equation 5. The point of equivalency occurs when the right-hand term equals D, so D′=D+D and D/D′=0.5). A few conclusions can be drawn from this:
(1) For small channels (r˜12 μm) the contribution from the velocity gradient dominates, D′>D, i.e. the contribution to dispersion from the velocity gradient is always large, especially for larger molecules which have smaller D; and
(2) For expansion channels (r>100 μm) the contribution from the velocity gradient is small for small molecules but remains large for larger molecules. This is a good result for this description, because it means that effects arising from radial diffusion of small molecules driven by the velocity gradient in a large diameter channel can reasonably be ignored. Thus, radial diffusion generated by depletion of compounds at the wall can be treated as a simple 1-D diffusion model.

As described earlier, a goal is to examine scaling relationships as the channel diameter is increased, including how axial flux, F, scales with radius. To simplify later derivations, equation 4 can be simplified to:

$${D}^{′} = D + \frac{{Q}^{2}}{Φ A D} = \frac{Φ A {D}^{2} + {Q}^{2}}{Φ A D} \qquad ( Eq . 6 )$$

where Φ=48π.

The axial flux of a molecule is given by Fick's Law (equation 2) into which we substitute the dispersion coefficient as given by equation 6:

F=D′AΔ (Eq. 7)

where Δ=dC/dx. Consider the situation in which flow begins in a first channel segment 1 and flows into a second channel segment 2. The axial flux at two points in the two segments can be examined as a ratio:

$$Ψ = \frac{{F}_{1}}{{F}_{2}} = \frac{{D}_{1}^{′} {A}_{1} {Δ}_{1}}{{D}_{2}^{′} {A}_{2} {Δ}_{2}} \qquad ( Eq . 8 )$$

where the subscripts 1 and 2 indicate channel segments 1 and 2, respectively. By continuity, as explained earlier, the axial concentration gradient becomes steeper when the channel diameter increases. It increases as the ratio of the areas (Equation 0), so equation 8 becomes:

$$Ψ = \frac{{F}_{1}}{{F}_{2}} = \frac{{D}_{1}^{′} {A}_{1} {Δ}_{1}}{{D}_{2}^{′} {A}_{2} ( {A}_{2} / {A}_{1} ) {Δ}_{1}} = \frac{{D}_{1}^{′} {A}_{1}^{2}}{{D}_{2}^{′} {A}_{2}^{2}} \qquad ( Eq . 9 )$$

The ratio of A₂ to A₁ is then set to k, such that:

A₂=kA₁ (Eq. 10)

and substituting this into equation 9 yields:

$$Ψ = \frac{{F}_{1}}{{F}_{2}} = \frac{{D}_{1}^{′} {A}_{1}^{2}}{{{D}_{2}^{′} ( {kA}_{1} )}^{2}} = \frac{{D}_{1}^{′} {A}_{1}^{2}}{{D}_{2}^{′} {k}^{2} {A}_{1}^{2}} = \frac{{D}_{1}^{′}}{{D}_{2}^{′} {k}^{2}} \qquad ( Eq . 11 )$$

Substituting equation 6 into equation 11 yields:

$$\begin{matrix}Ψ = \frac{{F}_{1}}{{F}_{2}} = \frac{{D}_{1}^{′}}{{D}_{2}^{′} k} \\ = ( \frac{Φ {D}^{2} {A}_{1} + {Q}^{2}}{Φ {DA}_{1}} ) ( \frac{ Φ {DA}_{2}}{Φ {D}^{2} {A}_{2} + {Q}^{2}} ) ( \frac{1}{{k}^{2}} ) \\ = ( \frac{Φ {D}^{2} {A}_{1} + {Q}^{2}}{Φ {DA}_{1}} ) ( \frac{Φ {DA}_{1} k}{Φ {D}^{2} {kA}_{1} + {Q}^{2}} ) ( \frac{1}{{k}^{2}} ) \\ = \frac{Φ {D}^{2} {A}_{1} + {Q}^{2}}{k ( Φ {D}^{2} {kA}_{1} + {Q}^{2} )}\end{matrix} \qquad ( Eq . 12 )$$

This is not a very clean equation. Basically, it states that k will have different effects on Ψ for different values of D, Q, and the first radius A₁.

The relative values of ΦD²A₁ and Q² will determine how the system behaves. If ΦD²A₁<<Q², then the solution approaches:

$$Ψ = \frac{{Q}^{2}}{{kQ}^{2}} = \frac{1}{k} \qquad ( Eq . 13 )$$

If ΦD²A₁>>Q², then the solution approaches:

$$Ψ = \frac{Φ {D}^{2} {A}_{1}}{{k}^{2} Φ {D}^{2} {A}_{1}} = \frac{1}{{k}^{2}} \qquad ( Eq . 14 )$$

The point of equivalency of flows for a small molecule (MW˜500) is:

$$Φ {D}^{2} {A}_{1} = Φ {D}^{2} π {r}^{2} = {Q}^{2} \\ {r}_{1}^{2} = \frac{{Q}^{2}}{Φ {D}^{2} π} = \frac{{Q}^{2}}{Φ {D}^{2} π} \\ {r}_{1} = \frac{Q}{π \sqrt{48} D} \qquad ( Eqn . 15 )$$

This is identical to equation 5, except for the subscript on the radius. This is an unexpected result. Equation 4 gives the radius where D′=2D, with larger radii giving D′→D and smaller radii giving D′>D. Equation 15 gives the segment 1 radius at which Equation 12 applies, with smaller radii giving

Ψ→1/k

Again, for flows with small molecules the point of equivalency is:

r₁=76.5 μm

This places one into the regime of ΦD²A₁<<Q² for an initial channel with radius r₁ of 12 μm so the relation Ψ=1/k applies. This says that if one increases the area of a channel by a factor of 10 (k=10) on entering an expansion, then the axial flux increases by 10 (F₂=10F₁). Put in terms of channel radius, if one increases the radius by a factor of 10 then one increases the axial flux by a factor of 100.

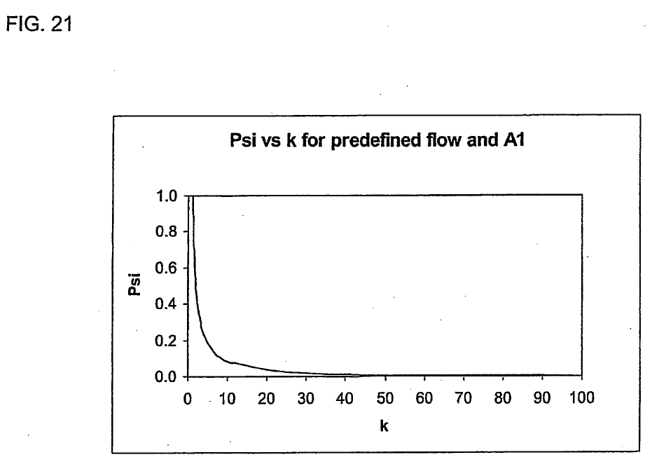

Rather than use these approximations, equation 12 can be used to calculate Ψ and plot this against different increasing k. This is done in FIG. 21 for the following exemplary conditions:

Q=30 nl/min=(30×10⁻¹² m³/60s)=5×10⁻¹³ m³/s

D=3×10⁻⁶ cm²s=3×10⁻¹⁰ m²/s

A₁=12 μm

As expected, it behaves approximately like 1/k (e.g. Ψ˜0.1 when k=10). Thus, two results are known:
Equation 4 (and the description following Equation 4) demonstrates that for expansion channels (r>100 μm) the contribution of the velocity gradient to the dispersion coefficient is small such that D′˜D in large diameter channels, such as for example the novel analysis channels disclosed herein. One implication of this result is that in large channels one should be able to approximate radial dispersion simply as radial diffusion. It also says that D′ in smaller channels is larger than D′ in larger channels:

r₁=12 μm then D′=2.25e−5 cm²/s

r₂=100 μm then D′=4.76e−6 cm²/s
(for D=3e−6 and Q=30 nL/min)
i.e. D′ is almost 5 times larger in the smaller channel.
Equation 15 (and the description following equation 15) indicates that the net axial flux in channels increases approximately linearly with the cross-sectional area A (radius squared) on transition to a large diameter channel. For the two channel radii given above, k=69.4 and Ψ=5.4e−3, i.e. F₂˜186 times greater than F₁ (Ψ calculated with the full equation 12, not equation 15). Thus, axial flux in the small channel relative to the large channel is more strongly influenced by the increase in cross-sectional area than the decrease in D′. For this example F₂ increases as 3 k ˜3r².

A goal at hand is to identify if increasing the diameter will cause radial diffusion to decrease more rapidly than axial flux increases. In other words, a determination is desired as to whether radial diffusion decreases with a power larger than 2. If not, a situation could exist in which the axial flux dissipates concentration gradients too quickly.

To summarize the above description:
(1) The diffusion distance from centerline to wall is larger in wider channels, as described above, so diffusive flux is slower;
(2) the surface area of wall per volume of fluid (the ratio of surface area to volume) becomes smaller such that the wall represents a smaller relative sink of compound;
(3) continuity of flow drives several related changes in the shape of a “unit” volume of fluid with the following effects:
   a. axial concentration gradients become steeper in larger diameter channels. Using continuity, the gradient becomes larger as

Δ₂=(A₂/A₁)Δ₁, where Δ=dC/dx; (Eq. 0)
b. radial concentration gradients become shallower in larger diameter channels, so diffusive flux is slower (this is related to the diffusion distance described in (1)); and
c. axial velocity becomes slower; and
(4) the velocity at the centerline V_{CL} (sometimes also known as Vₘₐₓ because the velocity is maximal at the centerline) becomes smaller, and the radial velocity gradient becomes shallower (dV/dr becomes smaller).

A description of the above summary of findings follows, staring with those points addressing axial flux.

Point 4: The analysis hereinabove permits dismissal of this issue this in large channels, such as for example the novel analysis channels disclosed herein. The velocity gradient contributes only minimally to dispersion in large channels.

Point 3a: The increase in the axial concentration gradient on transition into a larger channel dominates the transition, with ratio of axial flux in the small and large channels increasing varying with 1/k.

Point 3c. The axial velocity is given by Vₘₐₓ=2V. This will impact the timing of measurements (e.g. aging time and dilute time) if measurements are restricted to the centerline by confocal optics in a large channel; however, it will have no impact on measurements in a small channel. For example, aging time are currently estimated by dividing the volume of a segment of channel divided by the volumetric flow rate Q. The implicit assumption of this estimate is that the time of arrival is given by the average velocity V, not by the maximum velocity Vₘₐₓ, which is a reasonable assumption if radial concentration gradients are dissipated instantaneously in the channels. They are in small channels, but not in large channels.

The remaining summary points address radial diffusion.

Point 1: The diffusion distance increases linearly with r. The time required for a molecule to diffuse a given distance increases with the square of the distance, so flux to the wall should drop rapidly with increasing radius.

Point 2: As described in equation 0, the surface area per unit volume varies with 1/r. Thus, the rate of removal of compound at the wall, per unit volume, decreases with 1/r. For example, consider the situation in which one wants to watch a reaction for 3 minutes at 30 nl/min. The total volume in the channel is, thus, 90 nl. For exemplary 25 μm diameter channels, this yields a channel length of 0.144 m (14.4 cm). If one increases the diameter to 250 μm, then the length of channel enclosing 90 nl drops to 1.44 mm, or by a factor of 100. The surface area of the channel enclosing the 90 nl is 1.44e−5 m² and 1.44e−6 m² so the surface area is now 1/10th smaller, and the “sink” of compound in the larger channel is now 1/10^{th} that of the smaller channel.

Point 3b: The radial concentration gradient will become shallower, just as the axial concentration gets steeper, on transition into a larger channel. However, it will not increase linearly with r or with A, as the axial concentration gradient did, because of the geometry of the channel.

It will be understood that various details of the presently disclosed subject matter may be changed without departing from the scope of the disclosed subject matter. Furthermore, the foregoing description is for the purpose of illustration only, and not for the purpose of limitation.

### REFERENCES

Balasubramanian, V., Jayaraman, G., Iyengar, S. R. K. (1997). Effect of secondary flows on contaminant dispersion with weak boundary absorption. Applied Mathematical Modelling 21, 275-285.
Becker, H., Locascio, L. E. (2002). Polymer microfluidic devices. Talanta 56, 267-287.
Bevan, M. A., Prieve, D. C. (2000). Forces and hydrodynamic interactions between polystyrene surfaces with adsorbed PEO-PPO-PEO. Langmuir 16, 9274-9281.
Brandani, P., Stroeve, P. (2003). Adsorption and desorption of PEO-PPO-PEO triblock copolymers on a self-assembled hydrophobic surface. Macromolecules 36, 9492-9501.
Bridgett, M. J., Davies, M. C., Denyer, S. P. (1992). Control of staphylococcal adhesion to polystyrene surfaces by polymer surface modification with surfactants. Biomaterials 13, 411-6.
Bromberg, L., Salvati, L., Jr. (1999). Bioactive surfaces via immobilization of self-assembling polymers onto hydrophobic materials. Bioconjug. Chem. 10, 678-686.
De Cupere, V. M., Biltresse, S., Marchand-Brynaert, J., Rouxhet, P. G. (2003). Radiolabeling of Pluronic amphiphilic copolymer for adsorption studies. J. Colloid Interface Sci. 259, 163-170.
Desai, N. P., Hubbell, J. A. (1991). Solution technique to incorporate polyethylene oxide and other water-soluble polymers into surfaces of polymeric biomaterials. Biomaterials 12, 144-153.
Desai, N. P., Hubbell, J. A. (1992). Surface physical interpenetrating networks of poly(ethylene terephthalate) and poly(ethylene oxide) with biomedical applications. Macromolecules 25, 226-32.
Desai, N. P., Hossainy, S. F. A., Hubbell, J. A. (1992). Surface-immobilized polyethylene oxide for bacterial repellence. Biomaterials 13, 417-420.
Detrait, E., Lhoest, J. B., Bertrand, P., van den Bosch de Aguilar (1999). Fibronectin-pluronic coadsorption on a polystyrene surface with increasing hydrophobicity: relationship to cell adhesion. J. Biomed. Mater. Res. 45, 404-413.
Dewez, J. L., Berger, V., V, Schneider, Y. J., Rouxhet, P. G. (1997). Influence of Substrate Hydrophobicity on the Adsorption of Collagen in the Presence of Pluronic F68, Albumin, or Calf Serum. J. Colloid Interface Sci. 191, 1-10.
Dewez, J. L., Schneider, Y. J., Rouxhet, P. G. (1996). Coupled influence of substratum hydrophilicity and surfactant on epithelial cell adhesion. J. Biomed. Mater. Res. 30, 373-383.
Doherty, E. A., Meagher, R. J., Albarghouthi, M. N., Barron, A. E. (2003). Microchannel wall coatings for protein separations by capillary and chip electrophoresis. Electrophoresis 24, 34-54.
Green, R. J., Davies, M. C., Roberts, C. J., Tendler, S. J. (1998). A surface plasmon resonance study of albumin adsorption to PEO-PPO-PEO triblock copolymers. J. Biomed. Mater. Res. 42, 165-171.
Liu, V. A., Jastromb, W. E., Bhatia, S. N. (2002). Engineering protein and cell adhesivity using PEO-terminated triblock polymers. J. Biomed. Mater. Res. 60, 126-134.
Musoke, M., Luckham, P. E. (2004). Interaction forces between polyethylene oxide-polypropylene oxide ABA copolymers adsorbed to hydrophobic surfaces. Journal of Colloid & Interface Science 277, 62-70.
O'connor, S. M., Deanglis, A. P., Gehrke, S. H., Retzinger, G. S. (2000). Adsorption of plasma proteins on to poly(ethylene oxide)/poly(propylene oxide) triblock copolymer films: a focus on fibrinogen. Biotechnol. Appl. Biochem. 31 (Pt 3), 185-196.
Rossier, J. S., Gokulrangan, G., Girault, H. H., Svojanovsky, S., Wilson, G. S. (2000). Characterization of Protein Adsorption and Immunosorption Kinetics in Photoablated Polymer Microchannels. Langmuir 16, 8489-8494.
Tan, J. S., Butterfield, D. E., Voycheck, C. L., Caldwell, K. D., Li, J. T. (1993). Surface modification of nanoparticles by PEO/PPO block copolymers to minimize interactions with blood components and prolong blood circulation in rats. Biomaterials 14, 823-33.
Webb, K., Caldwell, K. D., Tresco, P. A. (2001). A novel surfactant-based immobilization method for varying substrate-bound fibronectin. J Biomed Mater Res 54, 509-18.
Yang, H. and Sundberg, S. A. Surface coating for microfluidic devices that incorporate a biopolymer resistant moiety. Technologies, Caliper. [U.S. Pat. No. 6,326,083]. 2001. USA. Dec. 4, 2001.

1. A microfluidic analysis channel, comprising:
a. an inlet having a first cross-sectional area for passage of fluid therethrough; and
b. an analysis region in fluid communication with the inlet and having a second cross-sectional area for passage of fluid from the inlet to the analysis region, the second cross-sectional area being greater than the first cross-sectional area, whereby adsorption of a compound in fluid in the analysis region is decreased and a reduction of concentration of the compound at a center axis region in the analysis region is minimized.

2. The analysis channel of claim 1, wherein the second cross-sectional area is at least two times greater than the first cross-sectional area.

3. The analysis channel of claim 2, wherein the second cross-sectional area is between about two times and about five hundred times larger than the first cross-sectional area.

4. The analysis channel of claim 1, wherein the analysis region has an aspect ratio of height to width equal to 1.

5. The analysis channel of claim 1, wherein the analysis channel further comprises an expansion region beginning at the inlet and having an upstream cross-sectional area approximately equivalent to the inlet first cross-sectional area and a downstream cross-sectional area approximately equivalent the second cross-sectional area.

6. The analysis channel of claim 5, wherein the downstream cross-sectional area is between about two times and about five hundred times larger than the upstream cross-sectional area.

7. The analysis channel of claim 1, wherein the analysis region further comprises a detection area located along at least a portion of a center axis region of the analysis region.

8. (canceled)

9. A microfluidic device, comprising:
a. at least one microscale channel for passage of fluid therethrough having a first cross-sectional area; and
b. an analysis channel in fluid communication with the microscale channel and having a second cross-sectional area, the second cross-sectional area being greater than the first cross-sectional area, whereby adsorption of a compound in fluid in the analysis channel is decreased and a reduction of concentration of the compound at a center axis region in the analysis channel is minimized.

10. The microfluidic device of claim 9, wherein the microfluidic device is comprised of a polymer, quartz, or silicon.

11. The microfluidic device of claim 9, wherein the analysis channel has an aspect ratio of height to width about equal to 1.

12. The microfluidic device of claim 9, wherein the second cross-sectional area is at least twice as large as the first cross-sectional area.

13. The microfluidic device of claim 12, wherein the second cross-sectional area is between about two times and about five hundred times larger than a cross-sectional area of the microscale channel.

14. The microfluidic device of claim 9, wherein the analysis channel further comprises an expansion region having an inlet in fluid communication with the microscale channel and an end opposite the inlet, the inlet having a cross-sectional area approximately equivalent to the first cross-sectional area of the microscale channel and the end having a cross-sectional area approximately equivalent to the second cross-sectional area of the analysis channel.

15. The microfluidic device of claim 14, wherein the cross-sectional area of the end is between about two times and about five hundred times greater than the cross-sectional area of the inlet.

16. The microfluidic device of claim 9, wherein the analysis channel comprises a detection area located along at least a portion of a center axis region of the analysis channel.

17. The microfluidic device of claim 9, comprising a controlled dispersion element in fluid communication with and located upstream of the analysis channel.

18. The microfluidic device of claim 17, wherein the controlled dispersion element is an expansion channel.

19-30. (canceled)

31. A method for making concentration dependent measurements in a microfluidic device, comprising:
a. flowing a fluid stream comprising at least one compound through at least one microscale channel of a microfluidic device;
b. continuously varying the concentration of the compound within the fluid stream;
c. flowing the fluid stream through an analysis channel in fluid communication with the microscale channel, the analysis channel comprising:
i. an inlet having a first cross-sectional area for passage of the fluid stream therethrough;
ii. an analysis region in fluid communication with the inlet and having a second cross-sectional area for passage of the fluid stream from the inlet to the analysis region, the second cross-sectional area being greater than the first cross-sectional area, whereby adsorption of the compound in the fluid stream in the analysis region is decreased and a reduction of concentration of the compound at a center axis region in the analysis region is minimized; and
iii. a detection area located within the analysis region; and
d. measuring the fluid stream at the detection area along at least a portion of the continuously varying concentration gradient of the compound.

32. The method of claim 31, wherein flowing the fluid stream comprising at least one compound through at least one microscale channel of the microfluidic device comprises a first compound flowing within a first fluid stream through a first microfluidic channel and a second compound flowing within a second fluid stream through a second microfluidic channel.

33. The method of claim 32, wherein the first and second microfluidic channels merge at a merge region, thereby flowing the first fluid stream into contact with the second fluid stream to form a merged fluid stream.

34. The method of claim 32, wherein continuously varying the concentration of the compound within the fluid stream comprises creating a continuous concentration gradient for the first and second compounds through controlled variation of volumetric flow rates of the first and second fluid streams.

35-57. (canceled)
